(19) United States
(12) Patent Application Publication
(10) Pub. No.: US 2022/0378949 A1
(43) Pub. Date: Dec. 1, 2022

(54) NEUROTROPHIN RECEPTOR BINDING CONJUGATE COMPOSITIONS, METHODS OF USE AND METHODS OF MAKING THEREOF

(71) Applicant: Manzanita Pharmaceuticals, Inc., Woodside, CA (US)

(72) Inventors: CONSTANCE A. MCKEE, Woodside, CA (US); STEPHEN B. KAHL, Woodside, CA (US); D. MICHAEL OLIVE, Lincoln, NE (US); GORDON CRAIG HILL, Whitestown, IN (US); LOUIS EUGENE BURTON, San Mateo, CA (US)

(21) Appl. No.: 17/769,693

(22) PCT Filed: Oct. 22, 2020
(86) PCT No.: PCT/US2020/056866

§ 371 Date: Apr. 15, 2022

Related U.S. Application Data
(60) Provisional application No. 62/924,965, filed on Oct. 23, 2019.
(60) Provisional application No. 62/967,828, filed on Jan. 30, 2020.
(60) Provisional application No. 62/976,725, filed on Feb. 14, 2020.

(51) Int. Cl. A61K 49/00; A61K 47/64; A61K 47/55; A61K 47/54; A61K 9/00

(52) U.S. Cl. CPC A61K 49/0056; A61K 47/642; A61K 47/55; A61K 47/545; A61K 9/0048; A61K 49/0058

(57) ABSTRACT

Neurotrophin receptor binding conjugate compositions for delivering an active agent to nerve cells are provided. Conjugate compositions of the present disclosure according to certain embodiments include a one or more active agent compounds (e.g., a dye or small molecule therapeutic) covalently bonded to a protein, peptide or peptidomimetic that binds selectively to a neurotrophin receptor. In embodiments of the compositions, the average ratio of active agent compound to protein, peptide or peptidomimetic in the conjugates is 5 or less. Dual color imaging compositions are also described. Methods for delivering an active agent conjugate selectively into nerve cells (e.g., intraocular delivery) are provided. Also included are methods of making compositions having neurotrophin receptor binding conjugates

### INTRODUCTION

Diseases of the peripheral nervous system (PNS) or central nervous system (CNS) may be caused by genetic defects in either one of the known low- (p75 neurotrophin receptor, p75NTR) or high-affinity (tropomyosin kinase, Trk) receptors, or neuronal degeneration due to chemical exposure e.g. chemotherapy, or trauma e.g. optic injury or optic nerve injury from blast. In addition, neurotrophins (NT) can function outside the nervous system. Many cells of the immune system and glia express the Trk family of receptors, including TrkA, the high affinity receptor associated with the NT Nerve Growth Factor (NGF).

The effectiveness of NGF as a pharmacologic agent to treat neurotrophic keratitis is consistent with evidence showing that NGF promotes survival and acts as an immunotrophin in a “complex bi-directional” function, possibly because TrkA and TrkB receptors, the high-affinity receptors associated with brain-derived neurotrophic factor (BDNF) are widely expressed throughout the immune system. This bi-directional interaction may explain emerging evidence that NGF plays a role in the crosstalk between neuro-immune-endocrine systems and also plays a complex role in modulating some cancers. In cancer, several signal transduction pathways have been identified as taking part in multiple NT functions, including the Ras/MAPK pathway, the PLCγ1 pathways, the PI3K/Akt-mTOR pathway, and the p75NTR-mediated signaling pathway, where intervention in one of more of those pathways may improve treatment of cancers of the nervous system and other organs.

### SUMMARY

Aspects of the present disclosure include neurotrophin receptor binding conjugate compositions for delivering an active agent to nerve cells. In certain embodiments, methods and compositions described herein include neurotrophin receptor conjugate compounds that bind a high affinity receptor. In some instances, the receptor may be physiologically and/or pathologically impaired. In some embodiments, the neurotrophin receptor conjugate compounds bind to a Trk receptor, such as for example TrkA receptors. In some embodiments, the neurotrophin receptor conjugate compounds bind to Trk receptors in a manner sufficient to facilitae endocytosis. In some embodiments, binding of the neurotrophin receptor conjugate compounds is sufficient to facilitate retrograde axonal transport of a dye or small molecule agent of the neurotrophin receptor conjugate compound. In some embodiments, binding of the neurotrophin receptor conjugate compounds is sufficient to facilitate anterograde axonal transport of a dye or small molecule agent of the neurotrophin receptor conjugate compound. In some embodiments, binding of the neurotrophin receptor conjugate compounds is sufficient to facilitate intracellular uptake of the dye or small molecule agent, such as, for example, uptake of the dye or small molecule agent into the neuronal cell body. In some embodiments, binding of the neurotrophin receptor conjugate compound is sufficient to facilitate intraneuronal transport of the the dye or small molecule agent. In some embodiments, binding of the neurotrophin receptor conjugate compound is sufficient to facilitate semi-intraneuronal transport of the dye or small molecule agent. In some embodiment, semi-intraneuronal transport of the dye or small molecule agent includes transport along the myelin nerve sheath. In some embodiments, uptake into the neuronal cell body is sufficient for the dye or small molecule agent to act intracellularly, such as, for example, where the dye exhibits fluorescence from the neuronal cell body or where the small molecule agent exhibits pharmacological activity. In certain embodiments, methods and compositions described herein include neurotrophin receptor conjugate compounds that exhibit one or more of: 1) binding to a receptor (e.g., a TrkA receptor); 2) retrograde or anterograde axonal transport of a dye or small molecule agent of the neurotrophin receptor conjugate compound; and 3) uptake of the dye or small molecule agent of the neurotrophin receptor conjugate compound into the neuronal cell body or semi-intraneuronal transport of the dye or small molecule agent such as transport along the myelin nerve sheath.

Neurotrophin receptor binding conjugate compositions according to certain embodiments include a one or more active agent compounds (e.g., a dye or small molecule therapeutic) covalently bonded to a protein, peptide or peptidomimetic that binds selectively to a neurotrophin receptor. In embodiments of the subject compositions, the average ratio of active agent compound to protein, peptide or peptidomimetic in the conjugates is 5 or less. In some embodiments, the average ratio of active agent compound to protein, peptide or peptidomimetic in the conjugates is 3.2 or less. In certain embodiments, the average ratio of the active agent component to the protein, peptide or peptidomimetic component is 2 or less, such as from about 0.95 to about 1.85. In some instances, 90% or more (e.g., 95% or more) of the active agent conjugates in the composition have an average ratio of active agent component to the protein, peptide or peptidomimetic component of 5 or less, such as where 90% or more (e.g., 95% or more) of the active agent conjugates in the composition have an average ratio of active agent component to the protein, peptide or peptidomimetic component of 3.2 or less, such as where 90% or more (e.g., 95% or more) of the active agent conjugates in the composition have an average ratio of active agent component to the protein, peptide or peptidomimetic component of 2 or less.

Aspects also include methods for intraocularly delivering an active agent to a nerve cell are provided. Methods according to certain embodiments include contacting an eye of a subject with a composition comprising an active agent conjugate, the active agent conjugate comprising one or more active agent compounds covalently bonded to a protein, peptide or peptidomimetic that binds selectively to a neurotrophin receptor. Conjugate compositions of the present disclosure are contacted with the surface of the eye (applied topically, e.g., to the corneal surface) or injected into the vitreous humor or implanted into the eye (where either the injection or implantation is described as intravitreal or intraocular administration). Contact lens and intravitreal implants containing the subject compositions are also described. The subject compositions also contemplate a chimeric protein of components of NGF that bind to TrkA and components of brain-derived neurotrophic factor (BDNF) that bind to TrkB, which chimera are used in a topical ocular application, to facilitate both trans-ocular transport and binding to TrkB receptors expressed in the back of the vitreous on the Retinal Ganglion Cells.

Aspects of the present disclosure further include compositions having two or more detectable label-biomolecule conjugates are provided. Compositions of the present disclosure according to certain embodiments, include a detectable label-biomolecule conjugate having a first detectable label covalently bonded to a protein, peptide or peptidomimetic that binds selectively to a neurotrophin receptor usually expressed on nervous tissue and a detectable label-biomolecule conjugate having a second detectable label covalently bonded to a biopolymer that binds selectively to neoplastic tissue. In certain embodiments, compositions of interest include a nerve imaging conjugate which is configured to image or structurally delineate a nerve and/or nerve processes and a tumor imaging conjugate which is configured to image a tumor. In certain instances, the subject compositions are used during surgery (e.g., resection surgery) where the nerve imaging conjugate and the tumor imaging conjugate are simultaneously administered to the subject and may be used as a visual aid during surgery, thus providing the oncologic surgeon with a visual surgical aid both to identify nerves and to identify individual cancer cells.

In embodiments, the protein, peptide or peptidomimetic component of conjugates in the subject compositions may be varied and may include, but is not limited to, nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF), and derivatives or fragments thereof. In some embodiments, the protein, peptide or peptidomimetic component of the subject conjugates are mammalian derived proteins, such as mammalian nerve growth factor, mammalian brain-derived neurotrophic factor or mammalian neurotrophic factor. In certain instances, the protein, peptide or peptidomimetic component of the subject conjugates are not bacterial derived proteins, such as E. coli-derived nerve growth factor, E. coli-derived brain-derived neurotrophic factor or E. coli-derived neurotrophic factor. In certain embodiments, conjugates of interest include a combination of two or more protein, peptide or peptidomimetic components, such as two or more selected from nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF), and derivatives or fragments thereof. In some embodiments, compositions of interest include a combination of two or more different conjugates, where the dye fluoresces in a different spectrum than the nerve-specific dye-NT conjugate, and where the cancer-specific dye conjugate uses a non-neurotrophin to bind to a specific non-Trk receptor. In some embodiments, the protein, peptide or peptidomimetic is a neurotrophic factor derived from a non-human animal. In other embodiments, the protein, peptide or peptidomimetic is a recombinant human neurotrophic factor. In still other embodiments, the protein, peptide or peptidomimetic is a subunit of a protein, such as a β-subunit of either NGF, rhNGF or rhBDNF, or a chimeric combination of one or more NTs. In some instances, the protein, peptide or peptidomimetic is a native neurotrophic factor (e.g., native NGF, native BDNF). In certain instances, the native neurotrophic factor does not include any additional peptide conjugates that have been functionalized for conjugating to a payload compound (e.g., dye or small molecule active agent). As described in greater detail below, neurotrophin binding conjugate compounds of the present disclosure according to certain embodiments include a native protein such as a native nerve growth factor (NGF), a native brain-derived neurotrophic factor (BDNF) or a native neurotrophic factor that is conjugated to the active agent compound through one or more amino acid residues (e.g., lysine residues) present in the native nerve growth factor (NGF), native brain-derived neurotrophic factor (BDNF) or native neurotrophic factor. In other words, according to these embodiments, the neurotrophin binding component does not include any non-natural or additional peptide sequences. In some instances, the protein, peptide or peptidomimetic component of subject conjugates do not include any additional C-terminal peptide sequences, such as for conjugating to a dye or active agent payload. In other instances, the protein, peptide or peptidomimetic component of subject conjugates do not include any additional N-terminal peptide sequences, such as for conjugating to a dye or active agent payload.

In certain embodiments, the protein, peptide or peptidomimetic is a neurotrophic factor that binds to tropomyosin kinase A (TrkA). In certain embodiments, the protein, peptide or peptidomimetic is a neurotrophic factor that exhibits retrograde axonal transport of a payload compound (e.g., dye or small molecule agent) conjugated to the protein, peptide or peptidomimetic. In certain embodiments, the protein, peptide or peptidomimetic is a neurotrophic factor that exhibits intraneuronal uptake, such as into the neuronal cell body, axon or dendrite of a payload compound (e.g., dye or small molecule agent) conjugated to the protein, peptide or peptidomimetic.

The active agent component of conjugates in the subject compositions is in certain instances a detectable label. Each detectectable label may be a compound such as fluorophore, chromophore, enzyme, redox label, radiolabels, acoustic label, Raman (SERS) tag, mass tag, isotope tag, magnetic particle, microparticle and nanoparticle. In some embodiments, each detectable label in the subject compositions is in certain instances, a dye such as an organic dye or an inorganic dye. In certain instances, the dye is a light emitting dye such as a fluorescent dye having an emission wavelength of 300 nm or more. Dyes of interest may include, but are not limited to, a bodipy dye, a coumarin dye, a rhodamine dye, an acridine dye, an anthraquinone dye, an arylmethane dye, a diarylmethane dye, a chlorophyll containing dye, a triarylmethane dye, an azo dye, a diazonium dye, a nitro dye, a nitroso dye, a phthalocyanine dye, a cyanine dye, an asymmetric cyanine dye, a quinon-imine dye, an azine dye, an eurhodin dye, a safranin dye, an indamin, an indophenol dye, a fluorine dye, an oxazine dye, an oxazone dye, a thiazine dye, a thiazole dye, a xanthene dye, a fluorene dye, a pyronin dye, a fluorine dye, a rhodamine dye, a phenanthridine dye, squaraines, bodipys, squarine roxitanes, naphthalenes, coumarins, oxadiazoles, anthracenes, pyrenes, acridines, arylmethines, or tetrapyrroles and a combination thereof. In certain embodiments, conjugates may include two or more dyes, such as two or more dyes selected from a bodipy dye, a coumarin dye, a rhodamine dye, an acridine dye, an anthraquinone dye, an arylmethane dye, a diarylmethane dye, a chlorophyll containing dye, a triarylmethane dye, an azo dye, a diazonium dye, a nitro dye, a nitroso dye, a phthalocyanine dye, a cyanine dye, an asymmetric cyanine dye, a quinon-imine dye, an azine dye, an eurhodin dye, a safranin dye, an indamin, an indophenol dye, a fluorine dye, an oxazine dye, an oxazone dye, a thiazine dye, a thiazole dye, a xanthene dye, a fluorene dye, a pyronin dye, a fluorine dye, a rhodamine dye, a phenanthridine dye, squaraines, bodipys, squarine roxitanes, naphthalenes, coumarins, oxadiazoles, anthracenes, pyrenes, acridines, arylmethines, or tetrapyrroles and a combination thereof. In some embodiments, conjugates in compositions of interest have an active agent component that includes a dye that is or is substantially the same as an Alexa Fluor dye, such as a dye that is or is substantially the same as an Alexa Fluor 488 dye or a dye that is or is substantially the same as an Alexa Fluor 680 dye or a dye that is or is substantially the same as an Alexa Fluor 780 dye. In other embodiments, conjugates in compositions of interest have an active agent component that includes a dye that is or is substantially the same as a Dyomics dye, such as a dye that is or is substantially the same as a Dyomics DY-800 dye. In yet other embodiments, conjugates in compositions of interest have an active agent component that includes a dye that is or is substantially the same as a Curadel dye, such as a dye that is or is substantially the same as a ZW-800 dye. In some embodiments, the dye is or is substantially the same as a Cy dye, such as Cy 7.5 or Cy 5.5. The dye is, in certain instances, a compound, having axial or planar symmetry. In some embodiments, the dye is a compound that facilitates intraneuronal visualization (e.g., with a spectrometer or with the naked eye) of the dye, where intraneuronal visualization may involve one or both well established forms of axonal transport, either the NT-Trk receptor vesicle in microtubules, and/or the NT-Trk receptor expressed in the myelin sheath. In some embodiments, axonal transport comprises retrograde axonal transport. In some embodiments, axonal transport comprises anterograde axonal transport. In these embodiments, conjugates of interest facilitate binding, endocytosis and intraneuronal transport (e.g., retrograde axonal transport or anterograde axonal transport) by a neuron or component thereof.

In other embodiments, the active agent component of conjugates in the subject compositions is a small molecule therapeutic active agent that acts intracellularly after synthetic manipulation, after Trk receptor binding and endocytosis, and after intraneuronal or retrograde/anteretograde axonal transport. In some instances, the small molecular therapeutic is an anti-cancer agent such as for the treatment of a cancer of the central nervous system, including a cancer selected from the group consisting of adult and pediatric gliomas, optic pathway glioma, spinal tumors, neurofibromatomas, schwannomas, malignant peripheral nerve sheath tumors, malignant schwannoma, neurofibrosarcoma, neurosarcoma. In some instances, the subject is diagnosed as having a glioma (low grade, high grade, etc.). In some embodiments, the small molecule therapeutic is a mammalian target of rapamycin (mTOR) inhibitor or mitogen-activated protein kinase (MEK) inhibitor. In other embodiments, the small molecule therapeutic is a compound selected from a glucocorticoid, a heat shock protein inhibitor, a checkpoint inhibitor and/or a chemokine/chemokine ligand inhibitor. For example, the glucocorticoid may be fluocinolone acetonide.

In some embodiments, biopolymers that bind selectively to neoplastic tissue are provided. The biopolymer that binds selectively to neoplastic tissue may be a polypeptide, a nucleic acid or a polysaccharide. In certain embodiments, the biopolymer component is a nucleic acid, such as an oligonucleotide, DNA or RNA. In other embodiments, the biopolymer component is a polypeptide, such as a peptidomimetic, a protein, an enzyme or an antibody. In certain embodiments, the biopolymer is an antibody that binds specifically to cancerous neoplastic tissue.

In some embodiments, the active agent component (e.g., detectable label such as a fluorescent dye) is associated with the protein, peptide or peptidomimetic component or biopolymer through a direct bond. In certain embodiments, the active agent component is covalently bonded directly to the protein, peptide or peptidomimetic component or biopolymer. In some embodiments, the active agent component is conjugated to the protein, peptide or peptidomimetic component through a bond to an internal amino acid residue of the protein, peptide or peptidomimetic. In other instances, the active agent component may be bonded to an intrachain amino acid residue along the surface of the protein, peptide or peptidomimetic. In still other instances, the active agent component may be bonded to a residue found at a binding site of the protein, peptide or peptidomimetic. In yet other instances, the active agent residue may be bonded to the protein, peptide or peptidomimetic component through an N-terminal amino acid or a C-terminal amino acid of the protein, peptide or peptidomimetic. In some embodiments, the active agent component is bonded to a mutated amino acid (e.g., amino acid substitution, non-natural amino acid, etc.) of the native protein, peptide or peptidomimetic. In other embodiments, the active agent component is bonded to a reactive amino acid of the native protein, peptide or peptidomimetic. For example, the linker may be bonded to a lysine residue of the protein, peptide or peptidomimetic. The active agent component may be bonded to a carbon or non-carbon atom of the protein, peptide or peptidomimetic. In one example, the active agent component is bonded to a carbon atom of the protein, peptide or peptidomimetic. In another example, the active agent component is bonded to a non-carbon atom of the protein, peptide or peptidomimetic, such as a nitrogen or sulfur atom of the protein, peptide or peptidomimetic.

In some embodiments, the active agent component (e.g., small molecule therapeutic) and the protein, peptide or peptidomimetic component or biopolymer may be associated together through one or more linkers. When present, the linker may be a cleavable linker or a non-cleavable linker. In some instances, the linker is a cleavable linker, such as an acid-cleavable linker, a base-cleavable linker, a photo-cleavable linker or an enzyme-cleavable (e.g., peptidase, esterase) linker. In certain instances, the linker includes a carbonate or carbmate moiety. In other instances, the linker is a non-cleavable linker. The linker may be a zero-length crosslinker, homobifunctional linker, heterobifunctional linker or a trifunctional crosslinker. In some embodiments, the linker may be used to conjugate the active agent component to the protein, peptide or peptidomimetic component through an internal amino acid residue of the protein, peptide or peptidomimetic. In other instances, the linker may be bonded to an intrachain amino acid residue along the surface of the protein, peptide or peptidomimetic. In still other instances, the linker may be bonded to a residue found in at a binding site of the protein, peptide or peptidomimetic. In yet other instances, the linker may be used to conjugate the active agent component to the protein, peptide or peptidomimetic component through an N-terminal amino acid or a C-terminal amino acid of the protein, peptide or peptidomimetic. In some embodiments, the linker is bonded to a mutated amino acid of the native protein, peptide or peptidomimetic. In other embodiments, the linker is bonded to a reactive amino acid of the native protein, peptide or peptidomimetic. The type of linkage for bonding the linker to the protein, peptide or peptidomimetic may be an ether linkage, a disulfide linkage or an amino linkage. For example, the linker may be bonded to a lysine residue of the protein, peptide or peptidomimetic. The linker may be bonded to a carbon or non-carbon atom of the protein, peptide or peptidomimetic. In one example, the linker is bonded to a carbon atom of the protein, peptide or peptidomimetic. In another example, the linker is bonded to a non-carbon atom of the protein, peptide or peptidomimetic, such as a nitrogen or sulfur atom of the protein, peptide or peptidomimetic.

Aspects of the present disclosure also include methods for delivering an active agent conjugate to a nerve cell by contacting the nerve cell (e.g., in vitro or in vivo) with a composition that includes a neurotrophin receptor binding conjugate having a one or more active agent compounds (e.g., a dye or small molecule therapeutic) covalently bonded to a protein, peptide or peptidomimetic that binds selectively to a neurotrophin receptor of the nerve cell. In embodiments, compositions contacted with the nerve cell have an average ratio of active agent compound to protein, peptide or peptidomimetic in the conjugates is 5 or less. In some embodiments, the average ratio of the active agent component to the protein, peptide or peptidomimetic is 3.2 or less. In certain embodiments, the average ratio of the active agent component to the protein, peptide or peptidomimetic component is 2 or less, such as from about 0.95 to about 1.85. In some instances, the nerve cell is contacted with a composition where 90% or more (e.g., 95% or more) of the active agent conjugates have an average ratio of active agent component to the protein, peptide or peptidomimetic component of 5 or less, such as where 90% or more (e.g., 95% or more) of the active agent conjugates in the composition have an average ratio of active agent component to the protein, peptide or peptidomimetic component of 3.2 or less, such as where 90% or more (e.g., 95% or more) of the active agent conjugates in the composition have an average ratio of active agent component to the protein, peptide or peptidomimetic component of 2 or less.

Aspects of the present disclosure also include methods for treating a subject with one or more of the subject compositions. Methods according to certain embodiments include administering to a subject a composition a neurotrophin receptor binding conjugate having a one or more active agent compounds (e.g., a dye or small molecule therapeutic) covalently bonded to a protein, peptide or peptidomimetic that binds selectively to a neurotrophin receptor of a nerve cell in the subject. In some instances, the composition is administered topically, intraocularly to the subject. In other instances, the composition is administered by injection, intracisternally to the subject. In yet other instances, the composition is administered by injection, intrathecally to the subject. In still other instances, the composition is administered intravitreally, either via intraocular injection or by intravitreal implant to the subject. In yet other instances, the composition is administered topically or transdermally to the subject. In other instances, the composition is administered to the subject by injection, such as by subcutaneous injection, intramuscular injection, or intrathecal injection.

Aspects of the present disclosure also include methods for preparing the subject compositions. Methods according to certain embodiments include contacting an active agent compound with a protein, peptide or peptidomimetic that binds selectively to a neurotrophin receptor to generate a composition having an active agent conjugate, the active agent conjugate comprising one or more active agent compounds covalently bonded to the protein, peptide or peptidomimetic that binds selectively to a neurotrophin receptor; and isolating (such as by reverse-phase high performance liquid chromatography, RP-HPLC) active agent conjugates from the composition having an average ratio of active agent compound to protein, peptide or peptidomimetic of 5 or less. In some embodiments, the average ratio of active agent compound to protein, peptide or peptidomimetic in the conjugates is 3.2 or less. In certain embodiments, compositions prepared by the subject methods have an average ratio of active agent compound to protein, peptide or peptidomimetic in the conjugates of 2 or less, such as from about 0.95 to about 1.85. In some instances, in compositions prepared by the subject methods 90% or more (e.g., 95% or more) of the active agent conjugates have an average ratio of active agent component to the protein, peptide or peptidomimetic component of 5 or less, such as where 90% or more (e.g., 95% or more) of the active agent conjugates in the composition have an average ratio of active agent component to the protein, peptide or peptidomimetic component of 3.2 or less, such as where 90% or more (e.g., 95% or more) of the active agent conjugates in the composition have an average ratio of active agent component to the protein, peptide or peptidomimetic component of 2 or less.

Aspects of the present disclosure also include methods for contacting a tissue of a subject (e.g., a human or non-human animal subject) with one or more of the subject compositions. In certain embodiments, the tissue of the subject (e.g., a human or non-human animal subject) contacted with the subject compositions is a neoplastic tissue. In other embodiments, the tissue of the subject contacted with the subject compositions is normal tissue. Methods according to certain embodiments include administering to a subject a composition that includes a first detectable label-biomolecule conjugate having a first detectable label covalently bonded to a protein, peptide or peptidomimetic that binds selectively to a neurotrophin receptor and a second detectable label-biomolecule conjugate having a second detectable label covalently bonded to a biopolymer that binds selectively to neoplastic tissue. In some instances, the composition is administered intraocularly, topically to the eye of the subject. In other instances, the composition is administered intracisternally to the subject. In yet other instances, the composition is administered intrathecally to the subject. In still other instances, the composition is administered intravitreally to the subject, which may be administered following intravitreal injection or implanted in the vitreous. In yet other instances, the composition is administered topically or transdermally to the subject. In other instances, the composition is administered to the subject by injection, such as by subcutaneous injection, intramuscular injection, intravitreal injection, intracisternal injection, or intrathecal injection.

### BRIEF DESCRIPTION OF THE FIGURES

The invention may be best understood from the following detailed description when read in conjunction with the accompanying drawings. Included in the drawings are the following figures:

FIG. 1A depicts fluorescent NGF in cell bodies after 10 ng/mL NGF-Alexa Fluor 488 (NGF-488) was applied at distal ends, where a form of murine-derived NGF was used (mNGF).

FIG. 1B depicts receptor mediated activity by adding 1000 ng/mL unlabeled NGF to 100 ng/mL mNGF-488.

FIG. 1C depicts neuronal uptake of mNGF-488 according to certain embodiments.

FIG. 2A-2D depict a construct with Dyomics DY-800 Near InfraRed (NIR) dye which was attached directly to recombinant human NGF (rhNGF) demonstrating that cell bodies can be visualized, indicating that the modified rhNGF compound has been retrogradedly transported.

FIG. 3 depicts that after synthetic manipulation, the TrkB binding activity of two constructs, NIR 800-rhBDNF where the NIR dye was directly attached to rhBDNF, and where the glucocorticoid fluocinolone acetonide (FA) was conjugated to rhBDNF using a heterobifunctional linker were assessed, both sustained neuronal survival.

FIG. 4 depicts analytics using mass spectroscopy (MS) to determine the active agent-peptide conjugate for sustained neuronal survival for different ratios of a fluorescent dye in the 800 region (800-rhNGF). The analytics quantify Dye-Adduct Ratio (DAR), or Drug-Adduct-Ratio (also DAR), which is an adaptation of a similar MS ratio analytic used to describe results from Antibody Drug Conjugates (ADCs). Leftmost panel is control, then F0, mixed or average=1.64 DAR; F1=0.96 DAR; F2=1.1 DAR; and F3=1.83 DAR.

FIG. 5 depicts reverse phase high performance liquid chromatography (RP-HPLC) analysis of synthesized active agent-peptide conjugate compositions according to certain embodiments.

FIG. 6 depicts analytics using mass spectroscopy (MS) to determine the active agent-peptide conjugate for sustained neuronal survival for different ratios of a fluorescent dye in the 800 region (800-rhBDNF).

### SELECT DEFINITIONS

The following terms have the following meaning unless otherwise indicated. Any undefined terms have their art recognized meanings.

“Pharmaceutical composition” refers to at least one compound and can further comprise a pharmaceutically acceptable carrier, with which the compound is administered to a patient.

“Pharmaceutically acceptable salt” refers to a salt of a compound, which possesses the desired pharmacological activity of the compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

The term “solvate” as used herein refers to a complex or aggregate formed by one or more molecules of a solute, e.g. a conjugate compound or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

“Pharmaceutically acceptable carrier” refers to a diluent, adjuvant, excipient or vehicle with, or in which a compound is administered.

“Preventing” or “prevention” or “prophylaxis” refers to a reduction in risk of occurrence of a condition, such as pain.

“Treating” or “treatment” of any condition, such as cancer, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically (e.g., stabilization of a discernible symptom), physiologically (e.g., stabilization of a physical parameter), or both. In certain embodiments, “treating” or “treatment” refers to delaying the onset of the condition.

“Therapeutically effective amount” means the amount of a compound (e.g., conjugate) that, when administered to a patient, is sufficient to effect such treatment. The “therapeutically effective amount” will vary depending on the compound, the condition and its severity, and the age, weight, etc., of the patient.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a compound refers to one or more compounds. As such, the terms “a”, “an”, “one or more” and“at least one” can be used interchangeably. Similarly, the terms “comprising,” “including,” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Compounds as described herein can be purified by any of the means known in the art, including chromatographic means, such as high performance liquid chromatography (HPLC), preparative thin layer chromatography, flash column chromatography, and ion exchange (IEX) chromatography. Any suitable stationary phase can be used, including normal and reversed phases as well as ionic resins. See, e.g., Introduction to Modem Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Representative Embodiments

Reference will now be made in detail to various embodiments. It will be understood that the invention is not limited to these embodiments. To the contrary, it is intended to cover alternatives, modifications, and equivalents as may be included within the spirit and scope of the allowed claims.

The present disclosure provides conjugate compositions, their methods of use and methods for making, where the conjugate compounds include an active agent (e.g., a dye or small molecule therapeutic) that is bonded either directly (e.g., to a detectable label such as a fluorescent dye) or through a linker (e.g., to a small molecule active agent) to a protein, peptide or pepetidomimetic that binds selectively to a neurotrophin receptor. In the subject compositions, the average ratio of active agent component to protein, peptide or peptidomimetic component in the conjugates is 5 or less (such as an average ratio of 3.2 or less, or in a composition such as an average ratio of 2 or less). Methods for using the conjugate compound compositions to selectively bind and deliver an active agent to a nerve cell such as by administering a therapeutically effective amount to a subject are also provided. Methods for making compositions having conjugate compounds where the average ratio of active agent component to protein, peptide or peptidomimetic component in the conjugates is 5 or less (such as an average ratio of 3.2 or less, such as an average ratio of 2 or less) are also described below.

The present disclosure also provides methods for intraocularly delivering an active agent to a nerve cell by contacting an eye of a subject with a composition comprising an active agent conjugate, the active agent conjugate comprising one or more active agent compounds covalently bonded to a protein, peptide or peptidomimetic that binds selectively to a neurotrophin receptor. The disclosure also provides pharmaceutical compositions which include one or more of the subject conjugate compounds and a pharmaceutically acceptable carrier and devices such as an intravitreal implant or contact lens device for delivering the subject compositions to the eye of the subject.

The present disclosure also provides compositions having a detectable label-biomolecule conjugate having a first detectable label covalently bonded to a protein, peptide or peptidomimetic that binds selectively to a neurotrophin receptor usually expressed at the distal end and/or nerve sheath of nerves comprising nervous tissue and a detectable label-biomolecule conjugate having a second detectable label covalently bonded to a biopolymer that binds selectively to neoplastic tissue.

### Active Agent Conjugate Compositions

As summarized above, aspects of the present disclosure include neurotrophin receptor binding conjugate compositions for delivering an active agent to nerve cells. Neurotrophin receptor binding conjugate compositions according to embodiments include a one or more active agent compounds (e.g., a dye or small molecule therapeutic, as described in greater detail below) covalently bonded to a protein, peptide or peptidomimetic in such a way that the compound binds selectively to a neurotrophin receptor and enables its intraneuronal transport, either via retrograde axonal transport and/or movement along the nerve sheath. In embodiments of the present disclosure, compositions of interest have a predetermined average ratio of active agent component to protein, peptide or peptidomimetic component. By “average ratio of active agent component to protein, peptide or peptidomimetic component” is meant the average number of active agents bonded to each protein, peptide or peptidomimetic in conjugates of the composition. In embodiments, compositions of interest have an average ratio of active agent component to protein, peptide or peptidomimetic component of 5 or less, such as an average ratio of 4.5 or less, such as 4.0 or less, such as 3.5 or less, such as 3.2 or less, such as 3.0 or less, such as 2.5 or less, such as 2.0 or less, such as 1.5 or less, such as 1.0 or less and including 0.5 or less. For instance, in one example where the active agent is a small molecule therapeutic, the average ratio of the small molecule therapeutic component to protein, peptide or peptidomimetic component in compositions of interest is 5.0 or less. In another example where the active agent is a dye, the average ratio of the dye component to protein, peptide or peptidomimetic component in compositions of interest is 2.0 or less. In some embodiments, the average ratio of active agent component to protein, peptide or peptidomimetic component in conjugates of the subject compositions ranges from 0.5 to 5.0, such as from 0.75 to 4.5, such as from 1.0 to 4.0, such as from 1.25 to 3.5 and including from 1.5 to 3.0. For instance, in one example, the average ratio of active agent component to protein, peptide or peptidomimetic component in conjugates of the composition is from about 0.95 to about 1.85.

In some embodiments, compositions of interest are purified such that 75% or more of the active agent conjugates in the composition have an average ratio of active agent component to protein, peptide or peptidomimetic component that is 5 or less, such as 80% or more, such as 85% or more, such as 90% or more, such as 95% or more, such as 97% or more, such as 99% or more, such as 99.9% or more, such as 99.99% or more and including 99.999% or more of the active agent conjugates in the composition have an average ratio of active agent component to protein, peptide or peptidomimetic component that is 5 or less. In certain embodiments, 75% or more of the active agent conjugates in the composition have an average ratio of active agent component to the protein, peptide or peptidomimetic component of 3.2 or less, such as 80% or more, such as 85% or more, such as 90% or more, such as 95% or more, such as 97% or more, such as 99% or more, such as 99.9% or more, such as 99.99% or more and including 99.999% or more of the active agent conjugates in the composition have an average ratio of active agent component to the protein, peptide or peptidomimetic component of 3.2 or less. In certain embodiments, 75% or more of the active agent conjugates in the composition have an average ratio of active agent component to the protein, peptide or peptidomimetic component of 2 or less, such as 80% or more, such as 85% or more, such as 90% or more, such as 95% or more, such as 97% or more, such as 99% or more, such as 99.9% or more, such as 99.99% or more and including 99.999% or more of the active agent conjugates in the composition have an average ratio of active agent component to the protein, peptide or peptidomimetic component of 2 or less.

In certain instances, the active agent is a small molecule therapeutic and 75% or more of the conjugates in the composition have an average ratio of small molecule therapeutic to protein, peptide or peptidomimetic that is 5 or less, such as 80% or more, such as 85% or more, such as 90% or more, such as 95% or more, such as 97% or more, such as 99% or more, such as 99.9% or more, such as 99.99% or more and including 99.999% or more of the conjugates in the composition have an average ratio of small molecule therapeutic to protein, peptide or peptidomimetic that is 5 or less. In other instances, the active agent is a small molecule therapeutic and 75% or more of the conjugates in the composition have an average ratio of small molecule therapeutic to protein, peptide or peptidomimetic that is 3.2 or less, such as 80% or more, such as 85% or more, such as 90% or more, such as 95% or more, such as 97% or more, such as 99% or more, such as 99.9% or more, such as 99.99% or more and including 99.999% or more of the conjugates in the composition have an average ratio of small molecule therapeutic to protein, peptide or peptidomimetic that is 3.2 or less. In other instances, the active agent is a small molecule therapeutic and 75% or more of the conjugates in the composition have an average ratio of small molecule therapeutic to protein, peptide or peptidomimetic that is 2 or less, such as 80% or more, such as 85% or more, such as 90% or more, such as 95% or more, such as 97% or more, such as 99% or more, such as 99.9% or more, such as 99.99% or more and including 99.999% or more of the conjugates in the composition have an average ratio of small molecule therapeutic to protein, peptide or peptidomimetic that is 2 or less.

In certain embodiments, the active agent is a dye and 75% or more of the conjugates in the composition have an average ratio of dye to protein, peptide or peptidomimetic that is 5 or less, such as 80% or more, such as 85% or more, such as 90% or more, such as 95% or more, such as 97% or more, such as 99% or more, such as 99.9% or more, such as 99.99% or more and including 99.999% or more of the conjugates in the composition have an average ratio of dye to protein, peptide or peptidomimetic that is 5 or less. In other instances, the active agent is a dye and 75% or more of the conjugates in the composition have an average ratio of dye to protein, peptide or peptidomimetic that is 3.2 or less, such as 80% or more, such as 85% or more, such as 90% or more, such as 95% or more, such as 97% or more, such as 99% or more, such as 99.9% or more, such as 99.99% or more and including 99.999% or more of the conjugates in the composition have an average ratio of dye to protein, peptide or peptidomimetic that is 3.2 or less. In other instances, the active agent is a dye and 75% or more of the conjugates in the composition have an average ratio of dye to protein, peptide or peptidomimetic that is 2 or less, such as 80% or more, such as 85% or more, such as 90% or more, such as 95% or more, such as 97% or more, such as 99% or more, such as 99.9% or more, such as 99.99% or more and including 99.999% or more of the conjugates in the composition have an average ratio of dye to protein, peptide or peptidomimetic that is 2 or less. For example, where the active agent is a dye, 75% or more of the conjugates in the composition have an average ratio of dye to protein, peptide or peptidomimetic that ranges from about 0.5 to about 2.0, such as from about 0.6 to about 1.9, such as from about 0.7 to about 1.8, such as from about 0.8 to about 1.7, such as from about 0.9 to about 1.6 and including from about 1.0 to about 1.5. In certain instances, the active agent is a dye and 75% or more of the conjugates in the composition have an average ratio of dye to protein, peptide or peptidomimetic that ranges from about 0.95 to about 1.85, such as 80% or more, such as 85% or more, such as 90% or more, such as 95% or more, such as 97% or more, such as 99% or more, such as 99.9% or more, such as 99.99% or more and including 99.999% or more of the conjugates in the composition have an average ratio of dye to protein, peptide or peptidomimetic that ranges from about 0.95 to about 1.85.

Conjugate compounds in the subject compositions include an active agent covalently bonded to a protein, peptide or pepetidomimetic that binds selectively to a neurotrophin receptor. In certain embodiments, compounds of interest include conjugates such as those depicted by formula: X−L−B, where X is an active agent such as a small molecule therapeutic or a dye; B is a protein, peptide or pepetidomimetic that binds selectively to a neurotrophin receptor; and L is an optional linker.

Conjugate compounds of interest include a protein, peptide or peptidomimetic that facilitates one or more of binding, endocytosis and transport (e.g., retrograde axonal transport to neuronal cell body) by a neurotrophin receptor. In some embodiments, the protein, peptide or peptidomimetic is a component that facilitates binding of the conjugate compound to a neurotrophin receptor. In other embodiments, the protein, peptide or peptidomimetic is a component that facilitates binding and endocytosis of the conjugate compound by a neurotrophin receptor. In yet other embodiments, the protein, peptide or peptidomimetic is a component that facilitates binding, endocytosis and transport (e.g., retrograde axonal transport, anterograde axonal transport) of the conjugate compound by a neurotrophin receptor. Protein, peptide or peptidomimetics of interest include, but are not limited to, nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF); derivatives, analogs, and fragments thereof such as recombinant molecules of NGF, BDNF, GDNF, CNTF, as well as synthetic peptides that bind to nerve cell surface receptors and have agonist or antagonist activities of growth factors. The protein, peptide or peptidomimetic may be derived from a non-human animal or may be recombinant human form expressed and produced either in bacterial systems e.g., E. coli cells or in mammalian cell systems, such as for example CHO (Chinese Hamster Ovary) cells. In some embodiments, the protein, peptide or peptidomimetic component of the subject conjugates are mammalian derived proteins, such as mammalian nerve growth factor, mammalian brain-derived neurotrophic factor or mammalian neurotrophic factor. In certain instances, the protein, peptide or peptidomimetic component of the subject conjugates are not bacterially derived proteins, such as E. coli-derived nerve growth factor, E. coli-derived brain-derived neurotrophic factor or E. coli-derived neurotrophic factor. In certain embodiments, the protein, peptide or peptidomimetic is a subunit of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF), such as a β-subunit of NGF, rhNGF or rhBDNF, or a chimeric combination of one or more NTs. In some instances, the protein, peptide or peptidomimetic is a native neurotrophic factor (e.g., native NGF, native BDNF). In certain instances, the native neurotrophic factor does not include any additional peptide conjugates that have been functionalized for conjugating to a payload compound (e.g., dye or small molecule active agent). As described in greater detail below, neurotrophin binding conjugate compounds of the present disclosure according to certain embodiments include a native protein such as a native nerve growth factor (NGF), a native brain-derived neurotrophic factor (BDNF) or a native neurotrophic factor that is conjugated to the active agent compound through one or more amino acid residues (e.g., lysine residues) present in the native nerve growth factor (NGF), native brain-derived neurotrophic factor (BDNF) or native neurotrophic factor. In other words, according to these embodiments, the neurotrophin binding component does not include any non-natural or additional peptide sequences. In some instances, the protein, peptide or peptidomimetic component of subject conjugates do not include any additional C-terminal peptide sequences, such as for conjugating to a dye or active agent payload. In other instances, the protein, peptide or peptidomimetic component of subject conjugates do not include any additional N-terminal peptide sequences, such as for conjugating to a dye or active agent payload.

In certain embodiments, the protein, peptide or peptidomimetic is a neurotrophic factor that binds tropomyosin kinase A (TrkA). In certain embodiments, the protein, peptide or peptidomimetic is a neurotrophic factor that exhibits retrograde axonal transport of a payload compound (e.g., dye or small molecule agent) conjugated to the protein, peptide or peptidomimetic. In certain embodiments, the protein, peptide or peptidomimetic is a neurotrophic factor that exhibits uptake (e.g., into the neuronal cell body or cellular processes) of a payload compound (e.g., dye or small molecule agent) conjugated to the protein, peptide or peptidomimetic.

Compositions of interest may include one or more types of protein, peptide or peptidomimetic conjugate compounds, such as two or more types, such as three or more types and including five or more types. For example, compositions may include one or more NGF-containing conjugates, BDNF-containing conjugates, NT-3-containing conjugates, NT-4/5-containing conjugates, NT-6-containing conjugates, NT-7-containing conjugates, GDNF-containing conjugates, CNTF-containing conjugates, or conjugates containing a chimeric construct of e.g. NGF-BDNF that preserves Trk binding activity after synthetic manipulation.

Conjugate compounds include one or more active agents. The term “active agent” is used herein to refer to compounds that are known to be active intracellularly, to be delivered to a nerve cell of interest, such as by endocytosis and/or transport (e.g., retrograde axonal transport to neuronal cell body) by the nerve cell. In some embodiments, the active agent is a small molecule compound such as a small molecule therapeutic. In other embodiments, the active agent is a dye. In some embodiments, the active agent is a dye where the binding to nerve tissue in the posterior of the eye may be used as a method to diagnose retinal disease, i.e., poor binding may reveal degeneration of retinal tissue. In certain embodiments, active agents in conjugates of interest are not biological macromolecules, such as a polysaccharide, a protein, nucleic acid or other biological macromolecule. In other embodiments, active agents in conjugates of interest are not polymers, such as a biological polymer. In still other embodiments, active agents in conjugates of interest have a molecular weight of 2000 g/mol or less, such as 1500 g/mol or less, such as 1000 g/mol or less, such as 900 g/mol or less, such as 750 g/mol or less, such as 500 g/mol or less, and including a molecular weight of 250 g/mol or less.

The active agent component of conjugates in the subject compositions is in certain instances a detectable label. Each detectectable label may be a compound such as fluorophore, chromophore, enzyme, redox label, radiolabels, acoustic label, Raman (SERS) tag, mass tag, isotope tag, magnetic particle, microparticle and nanoparticle. In some embodiments, each detectable label in the subject compositions is in certain instances, a dye such as an organic dye or an inorganic dye. In certain instances, the dye is a light emitting dye such as a fluorescent dye having a peak emission wavelength of 200 nm or more, such as 250 nm or more, such as 300 nm or more, such as 350 nm or more, such as 400 nm or more, such as 450 nm or more, such as 500 nm or more, such as 550 nm or more, such as 600 nm or more, such as 650 nm or more, such as 700 nm or more, such as 750 nm or more, such as 800 nm or more, such as 850 nm or more, such as 900 nm or more, such as 950 nm or more, such as 1000 nm or more and including 1050 nm or more. For example, the dye may be a fluorescent dye having a peak emission wavelength that ranges from 200 nm to 1200 nm, such as from 300 nm to 1100 nm, such as from 400 nm to 1000 nm, such as from 500 nm to 900 nm and including a fluorescent dye having a peak emission wavelength of from 600 nm to 800 nm.

Dyes of interest may include, but are not limited to, a bodipy dye, a coumarin dye, a rhodamine dye, an acridine dye, an anthraquinone dye, an arylmethane dye, a diarylmethane dye, a chlorophyll containing dye, a triarylmethane dye, an azo dye, a diazonium dye, a nitro dye, a nitroso dye, a phthalocyanine dye, a cyanine dye, an asymmetric cyanine dye, a quinon-imine dye, an azine dye, an eurhodin dye, a safranin dye, an indamin, an indophenol dye, a fluorine dye, an oxazine dye, an oxazone dye, a thiazine dye, a thiazole dye, a xanthene dye, a fluorene dye, a pyronin dye, a fluorine dye, a rhodamine dye, a phenanthridine dye, squaraines, bodipys, squarine roxitanes, naphthalenes, coumarins, oxadiazoles, anthracenes, pyrenes, acridines, arylmethines, or tetrapyrroles and a combination thereof. In certain embodiments, conjugates may include two or more dyes, such as two or more dyes selected from a bodipy dye, a coumarin dye, a rhodamine dye, an acridine dye, an anthraquinone dye, an arylmethane dye, a diarylmethane dye, a chlorophyll containing dye, a triarylmethane dye, an azo dye, a diazonium dye, a nitro dye, a nitroso dye, a phthalocyanine dye, a cyanine dye, an asymmetric cyanine dye, a quinon-imine dye, an azine dye, an eurhodin dye, a safranin dye, an indamin, an indophenol dye, a fluorine dye, an oxazine dye, an oxazone dye, a thiazine dye, a thiazole dye, a xanthene dye, a fluorene dye, a pyronin dye, a fluorine dye, a rhodamine dye, a phenanthridine dye, squaraines, bodipys, squarine roxitanes, naphthalenes, coumarins, oxadiazoles, anthracenes, pyrenes, acridines, arylmethines, or tetrapyrroles and a combination thereof.

In some embodiments, conjugates in compositions of interest have an active agent component that includes a dye that is or is substantially the same as an Alexa Fluor dye, such as a dye that is or is substantially the same as an Alexa Fluor 488 dye or a dye that is or is substantially the same as an Alexa Fluor 670 dye or a dye that is or is substantially the same as an Alexa Fluor 680 dye or a dye that is or is substantially the same as an Alexa Fluor 780 dye. In other embodiments, conjugates in compositions of interest have an active agent component that includes a dye that is or is substantially the same as a Dyomics dye, such as a dye that is or is substantially the same as a Dyomics DY-800 dye. In yet other embodiments, conjugates in compositions of interest have an active agent component that includes a dye that is or is substantially the same as a Curadel dye, such as a dye that is or is substantially the same as a ZW-800 dye. In some embodiments, the dye is or is substantially the same as a Cy dye, such as Cy 7.5 or Cy 5.5. The dye is, in certain instances, a compound, having axial or planar symmetry. In some embodiments, the dye is a compound that facilitates intraneuronal visualization (e.g., with a spectrometer or with the naked eye) of the dye. In these embodiments, conjugates of interest facilitate binding, endocytosis and intraneuronal transport (e.g., retrograde axonal transport to neuronal cell body) by a neuron or component thereof.

In some embodiments, conjugate compounds of interest include a small molecule compound, such as a small molecule therapeutic. In some instances, the small molecule therapeutic is an anti-cancer agent. In some instances, the cancer is a cancer of the central nervous system, such as a cancer selected from the group consisting of adult and pediatric gliomas (low grade glioma or a high grade glioma), optic pathway glioma, spinal tumors, neurofibromatomas, schwannomas, malignant peripheral nerve sheath tumors, malignant schwannoma, neurofibrosarcoma, and neurosarcoma. In certain instances, the cancer is optic pathway glioma. The anti-cancer agent may vary depending on the desired therapeutic effect and target indication and may be a mammalian target of rapamycin (mTOR) inhibitor or mitogen-activated protein kinase (MEK) inhibitor. In some embodiments, the anti-cancer agent is an mTOR inhibitor. For example, the anti-cancer agent may be sirolimus, temsirolimus, everolimus and ridaforolimus or a combination thereof. In other embodiments, the anti-cancer agent is an MEK inhibitor. For example, the anti-cancer agent may be trametinib, dabrafenib, cobimetinib, vemurafenib, binimetinib, selumetinib, or a combination thereof. In some instances, the anti-cancer agent is selumetinib. In some embodiments, the anti-cancer agent is a heat shock protein 90 (hsp90) inhibitor, such as alvespinomycin, 17-N-Allylamino-17-demethoxygeldanamycin (17AAG), luminespib (AUY-922, NVP-AUY922), ganetespib (STA-9090), onalespib (AT13387), NVP-BEP800, BIIB021, PF-04929113 (SNX-5422), SNX-2112 (PF-04928473), KW-2478, XL888, XL888, PU-H71, VER-49009, CH5138303, VER-50589, VER155008 and geldanamycin. In some embodiments, the anti-cancer agent is a checkpoint inhibitor. In some examples, the is an inhibitory compound that targets one or more of PD-1, PD-L1, CTLA4, TIM3, LAG3, VISTA, BTLA, TIGIT, LAIR1, CD160, 2B4 and TGFRβ, such as S7911, BMS202 and S8158. In some instances, the checkpoint inhibitor is an inhibitory compound that targets PD-1. In some embodiments, the anti-cancer agent is a chemokine 4/chemokine ligand 12 (CX4/CXCL12) inihibitor, such as burixafor, LY2510924, such as AMD3100 and AMD3465. In some embodiments, the compound is an imidazoquinolone amine. In certain embodiments, compositions of interest include one or more small molecule-containing conjugates as described in International Patent Application No. PCT/US2019/42253, filed on Jul. 17, 2019, the disclosure of which is incorporated herein by reference in its entirety.

In some embodiments, the active agent is an anti-inflammatory agent. In certain instances, the anti-inflammatory agent is a glucocorticoid, such as fluocinolone acetonide.

In other embodiments, the active agent is an antimicrobial compound. Antimicrobial agents of interest may include but are not limited to antibacterials, antifungals, antivirals, antiparasitics as well as antimicrobial pesticides. For example, antimicrobials may include fluoroquinolones such as ciprofloxacin, norfloxacin, ofloxacin, enoxacin, perfloxacin, fleroxacin, enrofloxacin, marbofloxacin, sarafloxacin, orbifloxacin, danofloxacin; aminoglycosides such as streptomycin, netilmicin, kanamycin, neomycin, tobramycin, amikacin, sisomicin, ribostamycin, dibekacin, framycetin, gentamycin, penicillins and aminopenicillins such as penicillin, ampicillin, amoxicillin, nafcillin, oxacillin and ticarcillin, cephalosporins such as ceftriaxone, cephalexin, cefadroxil and ceftiofur, β-lactams such as clavulanic acid which may be used in conjunction with penicillins or aminopenicillins, macrolides such as clarythromycin and erythromycin and other antibiotics such as dactinomycin, clindamycin, naladixic acid, chloramphenicol, rifamopin, clofazimine, spectinomycin, polymyxin B, colistin, minocycline, vancomycin, hygromycin B or C, fusidic acid, trimethoprim and cefotaxim. In some embodiments, antiviral compounds may include but are not limited to Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, T-705, and GS-5734 (remdesivir).

In some embodiments, the active agent component (e.g., detectable label such as a fluorescent dye) is associated with the protein, peptide or peptidomimetic component or biopolymer through a direct bond. In certain embodiments, the active agent component is covalently bonded directly to the protein, peptide or peptidomimetic component or biopolymer. In some embodiments, the active agent component is conjugated to the protein, peptide or peptidomimetic component through a bond to an internal amino acid residue of the protein, peptide or peptidomimetic. In other instances, the active agent component may be bonded to an intrachain amino acid residue along the surface of the protein, peptide or peptidomimetic. In still other instances, the active agent component may be bonded to a residue found at a binding site of the protein, peptide or peptidomimetic. In yet other instances, the active agent residue may be bonded to the protein, peptide or peptidomimetic component through an N-terminal amino acid or a C-terminal amino acid of the protein, peptide or peptidomimetic. In some embodiments, the active agent component is bonded to a mutated amino acid (e.g., substituted amino acid or non-natural amino acid) of the native protein, peptide or peptidomimetic. In other embodiments, the active agent component is bonded to a reactive amino acid of the native protein, peptide or peptidomimetic. For example, the active agent may be bonded to a lysine residue of the protein, peptide or peptidomimetic. The active agent component may be bonded to a carbon or non-carbon atom of the protein, peptide or peptidomimetic. In one example, the active agent component is bonded to a carbon atom of the protein, peptide or peptidomimetic. In another example, the active agent component is bonded to a non-carbon atom of the protein, peptide or peptidomimetic, such as a nitrogen or sulfur atom of the protein, peptide or peptidomimetic.

In certain embodiments, the active agent component is bonded to the protein, peptide or peptidomimetic component through a linker. The linker may be any convenient covalent linking protocol, such as a zero-length crosslinker, homobifunctional linker, heterobifunctional linker or a trifunctional crosslinker. The linker may include one or more functional groups, such as an amide, alkylamine, carbamate, carbonate, thiolether, alkyl, cycloalkyl or aryl moiety, as desired. In some embodiments, the linker includes a carbamate moiety. The linker may be used to conjugate the active agent component to the protein, peptide or peptidomimetic component through an N-terminal amino acid or a C-terminal amino acid of the protein, peptide or peptidomimetic. In some embodiments, the linker is bonded to a mutated amino acid of the native protein, peptide or peptidomimetic. The type of linkage for bonding the linker to the protein, peptide or peptidomimetic may be an ether linkage, a disulfide linkage or an amino linkage. For example, the linker may be bonded to a lysine residue of the protein, peptide or peptidomimetic. The linker may be bonded to a carbon or non-carbon atom of the protein, peptide or peptidomimetic. In one example, the linker is bonded to a carbon atom of the protein, peptide or peptidomimetic. In another example, the linker is bonded to a non-carbon atom of the protein, peptide or peptidomimetic, such as a nitrogen or sulfur atom of the protein, peptide or peptidomimetic.

In some embodiments, the linker is cleavable. The term “cleavable” is used herein in its conventional sense to refer to linkers that can be cleaved under predetermined conditions so as to break the bond between the active agent and the binding moiety. For example, the linker may be an acid-cleavable linker, a base-cleavable linker, a photo-cleavable linker or an enzyme-cleavable (e.g., peptidase, esterase) linker. Acid-cleavable linkers are cleaved by subjecting the conjugate compound to a pH of 7 or below, such as a pH of 6.5 or below, such as a pH of 6.0 or below, such as a pH of 5.5 or below, such as a pH of 5.0 or below, such as a pH of 4.5 of below, such as a pH of 4.0 or below, such as a pH of 3.5 or below, such as a pH of 3.0 or below, such as a pH of 2.5 or below, such a pH of 2.0 or below, such as a pH of 1.5 or below and including a pH of 1.0 or below. Base-cleavable linkers are cleaved by subjecting the conjugate compound to a pH of 7 or above, such as a pH of 7.5 or above, such as a pH of 8.0 or above, such as a pH of 8.5 or above, such as a pH of 9.0 or above, such as a pH of 9.5 of above, such as a pH of 10.0 or above, such as a pH of 10.5 or above, such as a pH of 11.0 or above, such as a pH of 11.5 or above, such a pH of 12.0 or above, such as a pH of 12.5 or above and including a pH of 13.0 or above.

In certain embodiments, the conjugate compounds in the subject compositions include an enzyme-cleavable linker. In some instances, the enzyme cleavable linker is cleaved by contacting the compound with a peptidase, such as trypsin or chymotrypsin. In other instances, the enzyme cleavable linker is cleaved by contacting the compound with an esterase. In some embodiments, linkers of interest include those described in U.S. Pat. Nos. 5,767,288; 5,563,250; 5,505,931 and 4,469,774, the disclosures of which are herein incorporated by reference.

In other embodiments, the conjugate compounds in the subject compositions include a non-cleavable linker. The term “non-cleavable” is used herein in its conventional sense to refer to a covalently bonded moiety that is stable under physiological conditions and does not release the active agent from the binding moiety (e.g., the dye or the small molecule therapeutic remains covalently bonded to the protein, peptide or peptidomimetic that selective binds the neurotrophin receptor). In other words, conjugate compounds having non-cleavable linkers are not susceptible to cleavage by acid, base, light or treatment with an enzyme. In these embodiments, 90% or more of conjugate compounds in a composition subjected to treatment by acid, base, light or with an enzyme does not result in release of the active agent from the binding moiety (e.g., protein, peptide or peptidomimetic that selective binds the nerve cells), such as 95% or more, such as 97% or more, such as 98% or more, such as 99% or more and including 99.9% or more of the conjugate compounds in a composition subjected to treatment by acid, base, light or with an enzyme does not result in release of the active agent from the binding moiety. Examples of suitable non-cleavable linkers may include, but are not limited to, maleimido-containing crosslinkers, such as: N-succinimidyl 4-(maleimidomethyl)cyclohexanecarboxylate (SMCC), N-succinimidyl-4-(N-maleimidomethyl)-cyclohexane-1-carboxy-(6-amidocaproate), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester [AMAS], succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI) or haloacetyl-containing crosslinkers, such as: N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido)propionate (SBAP).

In certain embodiments, compositions of interest include a pharmaceutically acceptable carrier. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Conjugate compositions having an average ratio of active agent component to protein, peptide or peptidomimetic component of 5 or less (e.g., 2 or less, such as 0.95 to 1.85) may be formulated into compositions suitable for delivery to a subject or for contacting with a nerve cell by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols, and/or formulated by lyophilizing

In certain instances, compositions of interest are formulated for injection such as by subcutaneous injection, intramuscular injection, intravitreal injection, intracisternal injection or intrathecal injection. In other instances, compositions are formulated to be administered intraocularly to the subject. In still other instances, compositions are formulated to be administered intracisternally to the subject. In yet other instances, compositions are formulated to be administered intrathecally to the subject. In still other instances compositions are formulated to be administered intravitreally to the subject. In yet other instances, compositions are formulated to be administered topically or transdermally to the subject.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interst further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection, the conjugate compounds may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Although the dosage used (as described in greater detail below) in treating a subject will vary depending on the clinical goals to be achieved, a suitable dosage range of the conjugate compound is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the route by which it is administered, where the effect of the neurotrophin as targeting moiety may localize the effect and thus affect the dose, the severity of the symptoms, and the susceptibility of the subject to side effects.

In some embodiments, a suitable dose of the conjugate compound in the subject compositions is in the range of from about 1 mg/kg body weight to about 500 mg/kg body weight, e.g., from about 5 mg/kg body weight to about 500 mg/kg body weight, from about 10 mg/kg body weight to about 500 mg/kg body weight, from about 20 mg/kg body weight to about 500 mg/kg body weight, from about 30 mg/kg body weight to about 500 mg/kg body weight, from about 40 mg/kg body weight to about 500 mg/kg body weight, from about 50 mg/kg body weight to about 500 mg/kg body weight, from about 60 mg/kg body weight to about 500 mg/kg body weight, from about 70 mg/kg body weight to about 500 mg/kg body weight, from about 80 mg/kg body weight to about 500 mg/kg body weight, from about 90 mg/kg body weight to about 500 mg/kg body weight, from about 100 mg/kg body weight to about 500 mg/kg body weight, from about 200 mg/kg body weight to about 500 mg/kg body weight, from about 300 mg/kg body weight to about 500 mg/kg body weight, or from about 400 mg/kg body weight to about 500 mg/kg body weight.

In some embodiments, a suitable dose of a compound in the subject compositions, is in the range of from about 1 mg/kg body weight to about 5 mg/kg body weight, from about 5 mg/kg body weight to about 10 mg/kg body weight, from about 10 mg/kg body weight to about 20 mg/kg body weight, from about 20 mg/kg body weight to about 30 mg/kg body weight, from about 30 mg/kg body weight to about 40 mg/kg body weight, from about 40 mg/kg body weight to about 50 mg/kg body weight, from about 50 mg/kg body weight to about 100 mg/kg body weight, or from about 100 mg/kg body weight to about 500 mg/kg body weight.

In some embodiments, a single dose of the conjugate compound is administered. In other embodiments, multiple doses of the conjugate compound are administered. Where multiple doses are administered over a period of time, the conjugate compound is administered, e.g., twice daily (qid), daily (qd), every other day (qod), every third day, three times per week (tiw), or twice per week (biw) over a period of time. For example, the conjugate compound is administered qid, qd, qod, tiw, or biw over a period of from one day to about 2 years or more. For example, the conjugate compound is administered at any of the aforementioned frequencies for one week, two weeks, one month, two months, six months, one year, or two years, or more, depending on various factors.

Dose units of the present disclosure can be made using manufacturing methods available in the art and can be of a variety of forms suitable for injection (including intracisternal, intrathecal, intravenous, intramuscular, subcutaneous, intravitreal and intraocular) administration, for example as a solution, suspension, solution, lyophilate or emulsion. The dose unit can contain components conventional in pharmaceutical preparations, e.g. one or more carriers, binders, lubricants, excipients (e.g., to impart controlled release characteristics in addition to any controlled release effects which may be contributed by conjugating the composition to a neurotrophin), pH modifiers, coloring agents or further active agents.

Dose units provided as liquid dose units can have a total weight of from about 1 microgram to about 1 gram, and can be from about 5 micrograms to 1.5 grams, from about 50 micrograms to 1 gram, from about 100 micrograms to 1 gram, from 50 micrograms to 750 milligrams, and may be from about 1 microgram to 2 grams.

Dose units can comprise components in any relative amounts. For example, dose units can be from about 0.1% to 99% by weight of active ingredients (i.e., conjugate compound) per total weight of dose unit. In some embodiments, dose units can be from 10% to 50%, from 20% to 40%, or about 30% by weight of active ingredients per total weight dose unit.

Dose units can be provided in a variety of different forms and optionally provided in a manner suitable for storage. For example, dose units can be disposed within a container suitable for containing a pharmaceutical composition. The container can be, for example, a bottle (e.g., with a closure device, such as a cap, a vial, an ampule (for single dose units), a dropper, thin film, a tube and the like.

Containers can include a cap (e.g., screw cap) that is removably connected to the container over an opening through which the dose units disposed within the container can be accessed.

Containers can include a seal which can serve as a tamper-evident and/or tamper-resistant element, which seal is disrupted upon access to a dose unit disposed within the container. Such seal elements can be, for example, a frangible element that is broken or otherwise modified upon access to a dose unit disposed within the container. Examples of such frangible seal elements include a seal positioned over a container opening such that access to a dose unit within the container requires disruption of the seal (e.g., by peeling and/or piercing the seal). Examples of frangible seal elements include a frangible ring disposed around a container opening and in connection with a cap such that the ring is broken upon opening of the cap to access the dose units in the container. In certain embodiments, the composition is a lyophilized composition and the subject conjugate compounds in the composition are reconsistituted at the point of care. In certain instances, the container is a reconstistute at the point of care pen-injectable.

Liquid dose units can be placed in a container (e.g., bottle or ampule) of a size and configuration adapted to maintain stability of dose units over a period during which the dose units are dispensed into a prescription. For example, containers can be sized and configured to contain 10, 20, 30, 40, 50, 60, 70, 80, 90, 100 or more single liquid dose units. The containers can be sealed or resealable. The containers can packaged in a carton (e.g., for shipment from a manufacturer to a pharmacy or other dispensary). Such cartons can be boxes, tubes, or of other configuration, and may be made of any material (e.g., cardboard, plastic, and the like). The packaging system and/or containers disposed therein can have one or more affixed labels (e.g., to provide information such as lot number, dose unit type, manufacturer, and the like).

The container can include a moisture barrier and/or light barrier, e.g., to facilitate maintenance of stability of the active ingredients in the dose units contained therein. The container can be adapted to contain a single dose unit or multiples of a dose unit. The container can include a dispensing control mechanism, such as a lock out mechanism that facilitates maintenance of dosing regimen.

Dose units can be provided in a container in which they are disposed, and may be provided as part of a packaging system (optionally with instructions for use). For example, dose units containing different amounts of the conjugate compounds can be provided in separate containers, which containers can be disposed with in a larger container (e.g., to facilitate protection of dose units for shipment). For example, one or more dose units as described herein can be provided in separate containers, where dose units of different compositions are provided in separate containers, and the separate containers disposed within package for dispensing.

### Dual Color Tissue Imaging Compositions

As summarized above, aspects of the present disclosure according to certain embodiments also include compositions having two or more active agent conjugate compounds, such as two or more detectable label-biomolecule conjugates. In these embodiments, compositions include a first detectable label-biomolecule conjugate having a first detectable label covalently bonded to a protein, peptide or peptidomimetic that binds selectively to a neurotrophin receptor. In some embodiments, compositions include: 1) a detectable label-biomolecule conjugate such as depicted by formula: X−L−A, where X is a detectable label; A is a protein, peptide or pepetidomimetic that binds selectively to a neurotrophin receptor; and L is an optional linker; and 2) a detectable label-biomolecule conjugate such as depicted by formula: X−L−B, where X is a detectable label; B is biopolymer (e.g., an antibody) that binds selectively to one or more receptors (e.g., non-Trk receptors) of the neoplastic tissue; and L is an optional linker.

Proteins, peptides or peptidomimetics of interest that bind selectively to a neurotrophin receptor according to certain embodiments facilitate one or more of binding, endocytosis and transport (e.g., retrograde axonal transport to neuronal cell body) by a neurotrophin receptor, such as those described above. For example, the proteins, peptides or peptidomimetics in active agent conjugate compounds of the subject dual dolor tissue imaging compositions may include, but are not limited to nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF); derivatives, analogs, and fragments thereof such as recombinant molecules of NGF, BDNF, GDNF, CNTF, as well as synthetic peptides that bind to nerve cell surface receptors and have agonist or antagonist activities of growth factors. The protein, peptide or peptidomimetic may be derived from a non-human animal or may be recombinant human. In certain embodiments, the protein, peptide or peptidomimetic is a subunit of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF), such as a β-subunit of NGF, rhNGF or rhBDNF, or a chimeric construct thereof, e.g. rhNGF-rhBDNF.

The subject dual dolor tissue imaging compositions include a detectable label-biomolecule conjugate that binds selectively to neoplastic tissue. The term “neoplastic tissue” is used herein in its conventional sense to refer to a tissue exhibiting an abnormal growth of cells. In some embodiments, the neoplastic tissue is benign. In other embodiments, the neoplastic tissue is malignant. The tissue may be any body tissue which can exhibit neoplastic growth, including but is not limited to brain and nervous system tissue, oral-esophogeal tissue, nasal tissue, muscle tissue, cardial and pericardial tissue, vascular tissue, lung tissue, splenic tissue, liver tissue, gall bladder tissue, pancreatic tissue, tissue of the gastrointestinal system, tissue of the genitourinary system as well as skin, bone, cartilaginous and ligament tissue.

The term “biopolymer” refers to a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes DNA (including cDNA), RNA and oligonucleotides, regardless of the source. As such, biomolecules may include polysaccharides, nucleic acids and polypeptides. For example, the nucleic acid may be an oligonucleotide, truncated or full-length DNA or RNA. In embodiments, oligonucleotides, truncated and full-length DNA or RNA are comprised of 10 nucleotide monomers or more, such as 15 or more, such as 25 or more, such as 50 or more, such as 100 or more, such as 250 or more and including 500 nucleotide monomers or more. For example, oligonucleotides, truncated and full-length DNA or RNA of interest may range in length from 10 nucleotides to 108 nucleotides, such as from 102 nucleotides to 107 nucleotides, including from 103 nucleotides to 106 nucleotides. In embodiments, biopolymers are not single nucleotides or short chain oligonucleotides (e.g., less than 10 nucleotides). By “full length,” it is meant that the DNA or RNA is a nucleic acid polymer having 70% or more of its complete sequence (such as found in nature), such as 75% or more, such as 80% or more, such as 85% or more, such as 90% or more, such as 95% or more, such as 97% or more, such as 99% or more and including 100% of the full length sequence of the DNA or RNA (such as found in nature) Polypeptides may be, in certain instances, truncated or full length proteins, enzyme or antibodies. In embodiments, polypeptides, truncated and full-length proteins, enzymes or antibodies are comprised of 10 amino acid monomers or more, such as 15 or more, such as 25 or more, such as 50 or more, such as 100 or more, such as 250 or more and including 500 amino acid monomers or more. For example, polypeptides, truncated and full-length proteins, enzymes or antibodies of interest may range in length from 10 amino acids to 108 amino acids, such as from 102 amino acids to 107 amino acids, including from 103 amino acids to 106 amino acids. In embodiments, biopolymers are not single amino acids or short chain polypeptides (e.g., less than 10 amino acids). By “full length,” it is meant that the protein, enzyme or antibody is a polypeptide polymer having 70% or more of its complete sequence (such as found in nature), such as 75% or more, such as 80% or more, such as 85% or more, such as 90% or more, such as 95% or more, such as 97% or more, such as 99% or more and including 100% of the full length sequence of the protein, enzyme or antibody (such as found in nature).

In some embodiments, the biopolymer is an antibody. Antibodies of interest encompass, but are not limited to, monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules can be fully human antibodies, humanized antibodies, or chimeric antibodies. The antibodies can include any antibody variable region, mature or unprocessed, linked to any immunoglobulin constant region. Minor variations in the amino acid sequences of antibodies or immunoglobulin molecules are encompassed by the present disclosure, providing that the variations in the amino acid sequence maintain 75% or more, e.g., 80% or more, 90% or more, 95% or more, or 99% or more of the sequence.

Antibody fragments can be a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen. “Fv” is the minimum antibody fragment which contains a complete antigen-recognition and -binding site. This region consists of a dimer of one heavy- and one light-chain variable domain in tight, non-covalent association. It is in this configuration that the three complimentarity-determining regions (CDRs) of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The “Fab” fragment also contains the constant domain of the light chain and the first constant domain (CHi) of the heavy chain. Fab fragments differ from Fab′ fragments by the addition of a few residues at the carboxyl terminus of the heavy chain CHi domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

Antibodies that can be used in connection with the present disclosure thus can encompass monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules can be fully human antibodies, humanized antibodies, or chimeric antibodies. In some embodiments, the antibody molecules are monoclonal, fully human antibodies.

The antibodies that can be used in connection with the present disclosure can include any antibody variable region, mature or unprocessed, linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

Minor variations in the amino acid sequences of antibodies or immunoglobulin molecules are encompassed by the present invention, providing that the variations in the amino acid sequence maintain at least 75%, e.g., at least 80%, 90%, 95%, or 99% of the sequence. In particular, conservative amino acid replacements are contemplated (e.g., as described herein). Whether an amino acid change results in a functional peptide can readily be determined by assaying the specific activity of the polypeptide derivative. Fragments (or analogs) of antibodies or immunoglobulin molecules, can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Sequence motifs and structural conformations can be used to define structural and functional domains in accordance with the invention.

In certain embodiments, the subject dual color imaging compositions include a detectable label-biomolecule conjugate having an antibody selected from Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Bevacizumab Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bivatuzumab, Brontictuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Panitumumab, Parsatuzumab, Pertuzumab, Pinatuzumab, Polatuzumab, Ranibizumab Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, Bevacizumab, Ranibizumab, Trastuzumab, Infliximab, Adalimumab, Efalizumab, Gemtuzumab ozogamicin, Tositumomab, Ibritumomab, Tiuxetan, Eculizumab, Alemtuzumab, Rituximab, Abiciximab, Cetuximab, Daclizumab, Basiliximab, Gemtuzumab, Natalizumab, Omalizumab, and Palivizumabor or an antigen-binding variant thereof. As used herein, the term “variant” refers to an antibody that binds to a particular cognate antigen but has fewer or more amino acids than the parental antibody, has one or more amino acid substitutions relative to the parental antibody, is a single-chain variant (such as an scFv variant) of the parental antibody, or any combination thereof.

As described in detail above, conjugates in the subject compositions include a detectable label where detetectable labels are moieties or markers that are detectable based on, for example, fluorescence emission, absorbance, fluorescence polarization, fluorescence lifetime, fluorescence wavelength, absorbance maxima, absorbance wavelength, Stokes shift, light scatter, mass, molecular mass, redox, acoustic, raman, magnetism, radio frequency, enzymatic reactions (including chemiluminescence and electro-chemiluminescence) or combinations thereof. In the subject dual color imaging compositions, the detectable label of each conjugate compound may be a fluorophore, chromophore, enzyme, redox label, radiolabels, acoustic label, Raman (SERS) tag, mass tag, isotope tag (e.g., isotopically pure rare earth element), magnetic particle, microparticle as well as a nanoparticle. In certain embodiments, the detectable label of each conjugate compound in the dual color imaging composition is a dye such as an organic dye or an inorganic dye (such as those described in detail above).

### Methods for Delivering an Active Agent to Nerve Cells

As summarized above, aspects of the present disclosure also include methods for delivering an active agent conjugate to a nerve cell by contacting the nerve cell (e.g., in vitro or in vivo) with a composition that includes a neurotrophin receptor binding conjugate having a one or more active agent compounds (e.g., a dye or small-molecule therapeutic) covalently bonded to a protein, peptide or peptidomimetic that binds selectively to a neurotrophin receptor of the nerve cell. As described above, the subject compositions also contemplate a chimeric protein of NGF that binds to TrkA and brain-derived neurotrophic factor (BDNF) that binds to TrkB, which chimera are used in a topical ocular application, to facilitate both trans-ocular transport and binding to TrkB receptors expressed in the back of the vitreous on the Retinal Ganglion Cells.

In embodiments, compositions contacted with the nerve cell have an average ratio of active agent compound to protein, peptide or peptidomimetic in the conjugates is 5 or less. In certain embodiments, the average ratio of the active agent component to the protein, peptide or peptidomimetic component is 3.2 or less, such as 2 or less, such as from about 0.95 to about 1.85. In some instances, the nerve cell is contacted with a composition where 90% or more (e.g., 95% or more) of the active agent conjugates have an average ratio of active agent component to the protein, peptide or peptidomimetic component of 5 or less, such as where 90% or more (e.g., 95% or more) of the active agent conjugates in the composition have an average ratio of active agent component to the protein, peptide or peptidomimetic component of 2 or less.

Aspects of the present disclosure also include methods for treating a subject with one or more of the subject compositions. In describing methods of the present disclosure, the term “subject” is meant the person or organism to which the conjugate compound is administered. As such, subjects of the present disclosure may include but are not limited to mammals, e.g., humans and other primates, such as chimpanzees and other apes and monkey species, dogs, rabbits, cats and other domesticated pets; and the like, where in certain embodiments the subject are humans. The term subject is also meant to include a person or organism of any age, weight or other physical characteristic, where the subjects may be an adult, a child, an infant, or a newborn. Methods according to certain embodiments include administering to a subject a composition a neurotrophin receptor binding conjugate having a one or more active agent compounds (e.g., a dye or small molecule therapeutic) covalently bonded to a protein, peptide or peptidomimetic that binds selectively to a neurotrophin receptor of a nerve cell in the subject. In some instances, the composition is administered intraocularly (e.g., topically such as on the surface or just below the surface of the eye or intravitreally) to the subject. In other instances, the composition is administered intracisternally to the subject. In yet other instances, the composition is administered intrathecally to the subject. In still other instances, the composition is administered intravitreally to the subject. In yet other instances, the composition is administered topically or transdermally to the subject. In other instances, the composition is administered to the subject by injection, such as by subcutaneous injection, intramuscular injection, intravitreal injection, intraocular injection, intracisternal injection or intrathecal injection. And in still other instances, the composition may be administered and exert a “controlled release” effect in nerve tissue due to either the pharmacologic properties of the drug, whether via direct delivery to the nerve cell body or by signalling effects, and/or the biologic properties of the neurotrophin, also via direct delivery via one or two possible transport mechanisms and/or via the ‘steady state’.

In certain embodiments, protocols may include multiple dosage intervals. By “multiple dosage intervals,” it is meant that two or more dosages of the conjugate compound composition are administered to the subject in a sequential manner. In practicing methods of the present disclosure, treatment regimens may include two or more dosage intervals, such as three or more dosage intervals, such as four or more dosage intervals, such as five or more dosage intervals, including ten or more dosage intervals.

The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer.

In certain embodiments, compositions of the invention can be administered prior to, concurrent with, or subsequent to other therapeutic agents for treating the same or an unrelated condition. If provided at the same time as another therapeutic agent, compositions having the subject conjugate compounds may be administered in the same or in a different composition. Thus, the anti-cancer-neurotropin binding conjugate compositions of interest and other therapeutic agents can be administered to the subject by way of concurrent therapy. By “concurrent therapy,” the intended administration of which to a subject is such that the therapeutic effect of the combination of the substances is caused in the subject undergoing therapy. For example, concurrent therapy may be achieved by administering the anti-cancer-neurotropin binding conjugate compositions of the invention with a pharmaceutical composition having at least one other agent, such as an anti-inflammatory agent, immunosuppressant, steroid, analgesic, anesthetic, antihypertensive, chemotherapeutic, among other types of therapeutics, which in combination make up a therapeutically effective dose, according to a particular dosing regimen. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

### Methods for Intraocularly Delivering an Active Agent Conjugate to Nerve Cells

As summarized above, aspects of the present disclosure also include methods for intraocularly delivering an active agent conjugate to nerve cells. Neurotrophin receptor binding conjugate compositions according to embodiments include a one or more active agent compounds (e.g., a dye or small molecule therapeutic, as described above) covalently bonded to a protein, peptide or peptidomimetic that binds selectively to a neurotrophin receptor. In practicing the subject methods according to this aspect, a composition having one or more of the conjugate compounds described herein is contacted with an eye of a subject. In some embodiments, the eye is contacted topically, such as by contacting the composition with the corneal surface of the eye. In some instances, the eye is topically contacted with the composition using a syringe, such as during a surgical procedure. In other embodiments, the composition is contacted to the eye using a contact lens that contains the subject compositions. In these embodiments, the contact lens may include a coating or the contact lens may incorporate directly a composition having one or more of the active agent conjugates described herein. In other embodiments, the composition is implanted into the eye of the subject, such as by intravitreal implantation. In these embodiments, the composition may be injected intravitreally into the eye, such as with a needle and syringe. In other embodiments, the composition may be administered intravitreally during a surgical procedure, such as during a vitrectomy procedure. In other embodiments, the composition is placed post-surgically, which may be an intravitreal procedure or a topical procedure or a combination thereof.

The composition may be contacted with the eye for a duration that varies, such as for 0.01 minute or more, such as for 0.05 minute or more, such as for 0.1 minute or more, such as for 0.5 minute or more, such as for 1 minute or more, such as for 5 minutes or more, such as for 10 minutes or more, such as for 15 minutes or more, such as for 30 minutes or more, such as for 60 minutes or more, such as for 2 hours or more, such as from 4 hours or more, such as for 6 hours or more, such as for 12 hours or more, such as for 24 hours or more and including for 168 hours or more.

In some embodiments, the composition is a sustained release composition that is configured to intraocularly deliver the active agent conjugate to the eye of the subject over a predetermined period of time, such as for 1 minute or more, such as for 5 minutes or more, such as for 10 minutes or more, such as for 15 minutes or more, such as for 30 minutes or more, such as for 60 minutes or more, such as for 2 hours or more, such as from 4 hours or more, such as for 6 hours or more, such as for 12 hours or more, such as for 24 hours or more and including for 168 hours or more. In certain embodiments, the composition is formulated to intraocularly deliver the active agent conjugate to the eye of the subject over 1 month or more.

In other embodiments, the composition is formulated as an immediate release composition is formulated to release 50% or more of the active agent conjugate or a pharmaceutically acceptable salt thereof within 10 minutes or less of administration of the composition to the subject, such as 60% or more, such as 75% or more, such as 90% or more, such as 95% or more and including 99% or more within 10 minutes or less of administration of the composition to the subject. In certain instances, the composition is formulated to release 50% or more of the active agent conjugate or a pharmaceutically acceptable salt thereof immediately after contacting the composition to the eye of the subject, such as 60% or more, such as 75% or more, such as 90% or more, such as 95% or more and including 99% or more immediately after contacting the composition to the eye of the subject.

In some embodiments, the subject compositions are formulated for delayed onset immediate release, an effect which may be due to one or more of the combination of several factors to include but not limited to formulation; to the direct delivery or indirect e.g. signalling properties of the small molecule therapeutic agent; and/or to the pharmacokinetics associated with the rate of absorption of the neurotrophin-Trk component of the composition. By delayed onset immediate release is meant that the composition is formulated to delay release of the active agent conjugates for a predetermined period of time after which, the active agent conjugate is immediately released. In some instances, the predetermined period of delay time may be 5 minutes or longer, such as 10 minutes or longer, such as 15 minutes or longer, such as 20 minutes or longer and including 30 minutes or longer. In some embodiments, the delayed onset immediate release composition is formulated such that 20% or less of the active agent conjugate in the composition is released approximately 20 minutes after contacting the eye of the subject and 75% or more of the active agent conjugate in the composition is released approximately 30 minutes thereafter. In these embodiments, 20% or less of the active agent conjugate in the composition is released approximately 20 minutes after administration to the subject, such as 15% or less, such as 10% or less, such as 5% or less, such as 3% or less and including 1% or less of the active agent conjugate is released approximately 20 minutes after administration to the subject. After the predetermined delay period (e.g., 20 minutes), the delayed onset immediate release active agent conjugate is formulated to release 75% or more of the active agent conjugate approximately 30 minutes thereafter, such as 80% or more, such as 85% or more, such as 90% or more, such as 95% or more, such as 97% or more and including 99% or more. In certain embodiments, after the predetermined delay period after administration, the composition is formulated to release 100% of the active agent conjugate.

In these embodiments, the subject compositions may be formulated to include one or more components which provide for the desired release profile. In certain embodiments, formulations include one or more non-erodible polymers. In certain instances, the subject compositions are coated with one or more polymers which provide for the desired release profile. In other embodiments, formulations include one or more bio-erodible polymers. For example, formulations may include one or more of a polymer such as cellulose, hydroxyethylcellulose, hydroxypropylcellulose, hydroxypropylmethylcellulose, carboxymethylcellulose, maltodextrin, sucrose, modified starch, a salt of alginic acid, soluble gums, carrageenan, polyvinylpyrrolidone (PVP) or polyvinylpolypyrrolidone (PVPP), ethylcellulose, cellulose acetate phthalate, hydroxypropylmethylcellulose phthalate, insoluble gums, polymethacrylate, a polyvinyl alcohol, shellac, and polyvinyl acetate phthalate and combinations thereof.

Although the dosage used in treating a subject will vary depending on the clinical goals to be achieved, a suitable dosage range of the conjugate compound is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility and/or sensitivity of the subject to the intended beneficial pharmacologic effects and/or to the intended or unintended side effects.

In some embodiments, a suitable dose of the active agent conjugate is in the range of from about 1 mg/kg body weight to about 500 mg/kg body weight, e.g., from about 5 mg/kg body weight to about 500 mg/kg body weight, from about 10 mg/kg body weight to about 500 mg/kg body weight, from about 20 mg/kg body weight to about 500 mg/kg body weight, from about 30 mg/kg body weight to about 500 mg/kg body weight, from about 40 mg/kg body weight to about 500 mg/kg body weight, from about 50 mg/kg body weight to about 500 mg/kg body weight, from about 60 mg/kg body weight to about 500 mg/kg body weight, from about 70 mg/kg body weight to about 500 mg/kg body weight, from about 80 mg/kg body weight to about 500 mg/kg body weight, from about 90 mg/kg body weight to about 500 mg/kg body weight, from about 100 mg/kg body weight to about 500 mg/kg body weight, from about 200 mg/kg body weight to about 500 mg/kg body weight, from about 300 mg/kg body weight to about 500 mg/kg body weight, or from about 400 mg/kg body weight to about 500 mg/kg body weight.

In some embodiments, a suitable dose of the active agent conjugate, is in the range of from about 1 mg/kg body weight to about 5 mg/kg body weight, from about 5 mg/kg body weight to about 10 mg/kg body weight, from about 10 mg/kg body weight to about 20 mg/kg body weight, from about 20 mg/kg body weight to about 30 mg/kg body weight, from about 30 mg/kg body weight to about 40 mg/kg body weight, from about 40 mg/kg body weight to about 50 mg/kg body weight, from about 50 mg/kg body weight to about 100 mg/kg body weight, or from about 100 mg/kg body weight to about 500 mg/kg body weight.

In some embodiments, a single dose of the active agent conjugate is administered. In other embodiments, multiple doses of the conjugate compound are administered. Where multiple doses are administered over a period of time, the conjugate compound is administered, e.g., twice daily (qid), daily (qd), every other day (qod), every third day, three times per week (tiw), or twice per week (biw) over a period of time. For example, the conjugate compound is administered qid, qd, qod, tiw, or biw over a period of from one day to about 2 years or more. For example, the conjugate compound is administered at any of the aforementioned frequencies for one week, two weeks, one month, two months, six months, one year, or two years, or more, depending on various factors.

In certain embodiments, protocols may include multiple dosage intervals. By “multiple dosage intervals” is meant that two or more dosages of the conjugate compound composition is administered to the subject in a sequential manner. In practicing methods of the present disclosure, treatment regimens may include two or more dosage intervals, such as three or more dosage intervals, such as four or more dosage intervals, such as five or more dosage intervals, including ten or more dosage intervals.

The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer. In certain embodiments, the subject compositions are formulated to deliver the active agent conjugate to the subject (e.g., through implantation, such as an intravitreal implantation) for a prolonged period of time, such as for 3 months or longer, such as for 6 months or longer, such as 9 months or longer, such as for 1 year or longer, such as for 5 years or longer and including for 10 years or longer.

### Methods for Administering a Dual Color Imaging Composition

Aspects of the present disclosure also include methods for contacting a tissue of a subject with one or more dual color imaging compositions as described above. In certain embodiments, the tissue of the subject contacted with the subject compositions is a neoplastic tissue. In other embodiments, the tissue of the subject contacted with the subject compositions is normal tissue. In certain embodiments, the tissue contacted is ocular tissue. Methods according to certain embodiments include administering to a subject a composition that includes a first detectable label-biomolecule conjugate having a detectable label covalently bonded to a protein, peptide or peptidomimetic that binds selectively to a neurotrophin receptor and a detectable label-biomolecule conjugate having a second detectable label covalently bonded to a biopolymer that binds selectively to neoplastic tissue. In some instances, the composition is administered topically, intra-operatively to the subject. In other instances, the composition may be administered intraocularly to the subject. In other instances, the composition is administered intracisternally to the subject. In yet other instances, the composition is administered intrathecally to the subject. In still other instances, the composition is administered intravitreally to the subject. In yet other instances, the composition is administered topically or transdermally to the subject. In other instances, the composition is administered to the subject by injection, such as by subcutaneous injection, intramuscular injection, intravitreal injection, intracisternal injection, or intrathecal injection.

In some embodiments, the subject compositions provide for a dual color imaging of nervous tissue and neoplastic tissue, such as during a surgical procedure. In certain instances, the surgical procedure is a tissue resection procedure, e.g., tumoral tissue resection surgery. In certain embodiments, methods include administering to the subject undergoing surgery a composition that includes a first detectable label-biomolecule conjugate having a detectable label covalently bonded to a protein, peptide or peptidomimetic that binds selectively to a neurotrophin receptor and a detectable label-biomolecule conjugate having a second detectable label covalently bonded to a biopolymer that binds selectively to a receptor of the neoplastic tissue and detecting the nerve imaging component and the neoplastic tissue imaging component. The imaging components of the nerve imaging component and the neoplastic tissue imaging component may be visualized by the same or different protocols. For example, the imaging components of the nerve imaging component and the neoplastic tissue imaging component may be each independently visualized by the naked eye or by spectroscopic detection, e.g., ultraviolet light detection, infrared or near infrared (NIR) light detection, or in a fluorescent spectrum below the NIR spectrum.

In some embodiments, the composition is administered to the subject before the surgical procedure, such as 5 minutes or more, such as 10 minutes or more, such as 15 minutes or more, such as 30 minutes or more, such as 60 minutes or more and including 120 minutes or more. In certain embodiments, the composition is administered to the subject during the surgical procedure.

### Methods for Preparing Conjugate Compound Compositions

Aspects of the present disclosure also include methods for preparing the subject compositions. Methods according to certain embodiments include contacting an active agent compound with a protein, peptide or peptidomimetic that binds selectively to a neurotrophin receptor to generate a composition having an active agent conjugate, the active agent conjugate comprising one or more active agent compounds covalently bonded to the protein, peptide or peptidomimetic that binds selectively to a neurotrophin receptor; and isolating active agent conjugates from the composition having an average ratio of active agent compound to protein, peptide or peptidomimetic of 5 or less. In certain embodiments, compositions prepared by the subject methods have an average ratio of active agent compound to protein, peptide or peptidomimetic in the conjugates of 2 or less, such as from about 0.95 to about 1.85. In some instances, in compositions prepared by the subject methods 90% or more (e.g., 95% or more) of the active agent conjugates have an average ratio of active agent component to the protein, peptide or peptidomimetic component of 5 or less, such as where 90% or more (e.g., 95% or more) of the active agent conjugates in the composition have an average ratio of active agent component to the protein, peptide or peptidomimetic component of 2 or less.

In certain embodiments, methods include contacting an active agent with a linker precursor to produce an activated active agent and contacting the activated active agent with a protein, peptide or pepetidomimetic that binds selectively to a neurotrophin receptor. In embodiments, the linker precursor may be a zero-length crosslinker precursor, homobifunctional linker precursor, heterobifunctional linker precursor or a trifunctional crosslinker precursor. In certain embodiments, the active agent is contacted with a bifunctional linker precursor to produce the activated active agent, such as a homobifunctional or heterobifunctional linker precursor. In some embodiments, the bifunctional linker precursor includes a succimide, such as where the the bifunctional linker precursor is N,N′-disuccinimidyl carbonate. The linker precursor is contacted with the active agent and with a reactive moiety of active agent. The linker may be used to conjugate the active agent component to the protein, peptide or peptidomimetic component through an N-terminal amino acid or a C-terminal amino acid of the protein, peptide or peptidomimetic. In some embodiments, the linker is bonded to a mutated amino acid of the native protein, peptide or peptidomimetic. The type of linkage for bonding the linker to the protein, peptide or peptidomimetic may be an ether linkage, a disulfide linkage or an amino linkage. For example, the linker may be bonded to a lysine residue of the protein, peptide or peptidomimetic. The linker may be bonded to a carbon or non-carbon atom of the protein, peptide or peptidomimetic. In one example, the linker is bonded to a carbon atom of the protein, peptide or peptidomimetic. In another example, the linker is bonded to a non-carbon atom of the protein, peptide or peptidomimetic, such as a nitrogen or sulfur atom of the protein, peptide or peptidomimetic. The reactive group may be a nucleophilic or electrophilic reactive group, depending on the reactive group of the linker precursor. In some instances, the active agent includes a nucleophilic reactive group, such as a hydroxyl group. In certain instances, the active agent includes a primary hydroxyl group. Methods according to certain embodiments include functionalizing the active agent to include a reactive group, such as a nucleophilic reactive group (e.g., hydroxyl group) for reacting with the linker precursor component. Coupling of the linker precursor with the active agent produces an activated active agent. The active agent is contacted with the protein, peptide or pepetidomimetic that selectively binds to the nerve cell (e.g., neurotrophin receptor) to produce the conjugate compounds of the subject compositions.

Methods for preparing the subject compositions include isolating active agent conjugates from the composition. In some embodiments, isolating the active agent conjugates includes separating the active agent conjugates of interest by reverse phase high performance liquid chromatography (RP-HPLC). In certain embodiments, methods include isolating active agent conjugates from the composition having an average ratio of active agent compound to protein, peptide or peptidomimetic of 5 or less. In some embodiments, methods include purifying the composition to generate a composition having conjugates having a predetermined ratio of active agent compound to protein, peptide or peptidomimetic, such as 3.2 or less, such as 2 or less, such as from about 0.95 to about 1.85. In some instances, methods include purifying to generate a composition where 90% or more (e.g., 95% or more) of the active agent conjugates have an average ratio of active agent component to the protein, peptide or peptidomimetic component of 5 or less, such as where 90% or more (e.g., 95% or more) of the active agent conjugates in the composition have an average ratio of active agent component to the protein, peptide or peptidomimetic component of 3.2 or less, such as where 90% or more (e.g., 95% or more) of the active agent conjugates in the composition have an average ratio of active agent component to the protein, peptide or peptidomimetic component of 2 or less. The term “purified” is used in its conventional sense to refer to a composition where at least some isolation or purification process has been conducted, such as for example, filtration or aqueous workup of a reaction mixture. In certain instances, purification includes liquid chromatography, recrystallization, distillation (e.g., azeotropic distillation) or other type of compound purification.

### ASPECTS OF THE DISCLOSURE

Aspects, including embodiments, of the subject matter described herein may be beneficial alone or in combination, with one or more other aspects or embodiments. Without limiting the description, certain non-limiting aspects of the disclosure numbered 1-194 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A composition comprising an active agent conjugate, the active agent conjugate comprising one or more active agent compounds covalently bonded to a protein, peptide or peptidomimetic that binds selectively to a neurotrophin receptor, is endocytosed and enables intracellular transposition (e.g., retrograde axonal transport),

wherein the average ratio of active agent compound to protein, peptide or peptidomimetic in the active agent conjugates in the composition is 5 or less.

2. The composition according to 1, wherein 95% or more of the active agent conjugates in the composition have an average ratio of active agent compound to protein, peptide or peptidomimetic of 5 or less.
3. The composition according to any one of 1-2, wherein the average ratio of active agent compound to protein, peptide or peptidomimetic in the active agent conjugates in the composition is 3.2 or less.
4. The composition according to any one of 1-2, wherein the average ratio of active agent compound to protein, peptide or peptidomimetic in the active agent conjugate in the composition is 2 or less.
5. The composition according to 4, wherein the ratio of active agent compound to protein, peptide or peptidomimetic in the active agent conjugate is from about 0.95 to about 1.85.
6. The composition according to 4, wherein 90% or more of the active agent conjugates in the composition have an average ratio of active agent compound to protein, peptide or peptidomimetic of 2 or less.
7. The composition according to 4, wherein 95% or more of the active agent conjugates in the composition have an average ratio of active agent compound to protein, peptide or peptidomimetic of 2 or less.
8. The composition according to any one of 1-7, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.
9. The composition according to claim 8, wherein the protein, peptide or peptidomimetic is a neurotrophic factor derived from a non-human animal.
10. The composition according to 8, wherein the protein, peptide or peptidomimetic is a recombinant human neurotrophic factor.
11. The composition according to 8, wherein the protein, peptide or peptidomimetic comprises a $-subunit of NGF, rhNGF or rhBDNF, or a chimeric construct of rhNGF and rhBDNF, or a chimeric construct of rhNGF and any other neurotrophic factor, or a chimeric construct between any of two other neurotrophic factors.
12. The composition according to any one of 1-11, wherein the active agent is a dye.
13. The composition according to 12, wherein the active agent is an organic dye or an inorganic dye.
14. The composition according to 12, wherein the dye is selected from the group consisting of a bodipy dye, a coumarin dye, a rhodamine dye, an acridine dye, an anthraquinone dye, an arylmethane dye, a diarylmethane dye, a chlorophyll containing dye, a triarylmethane dye, an azo dye, a diazonium dye, a nitro dye, a nitroso dye, a phthalocyanine dye, a cyanine dye, an asymmetric cyanine dye, a quinon-imine dye, an azine dye, an eurhodin dye, a safranin dye, an indamin, an indophenol dye, a fluorine dye, an oxazine dye, an oxazone dye, a thiazine dye, a thiazole dye, a xanthene dye, a fluorene dye, a pyronin dye, a fluorine dye, a rhodamine dye, a phenanthridine dye, squaraines, bodipys, squarine roxitanes, naphthalenes, coumarins, oxadiazoles, anthracenes, pyrenes, acridines, arylmethines, or tetrapyrroles and a combination thereof.
15. The composition according to 12, wherein the dye has an emission wavelength of 300 nm or more.
16. The composition according to 12, wherein the dye is Alexa Fluor 488.
17. The composition according to 12, wherein the dye is Dyomics DY-800.
18. The composition according to 12, wherein the dye is ZW-800.
19. The composition according to 12, wherein the dye is a compound having axial symmetry.
20. The composition according to 12, wherein the dye is a compound having a plane of symmetry.
21. The composition according to 12, wherein the dye is a compound that facilitates intraneuronal visualization of the dye, where intraneuronal visualization may be due to Trk receptor binding, endocytosis and retrograde axonal transport either in microtubules and/or in the nerve sheath encircling a nerve.
22. The composition according to any one of 1-11, wherein the active agent is a small molecule active agent.
23. The composition according to any one of 1-11, wherein the active agent is an anti-cancer agent.
24. The composition according to any one of 1-11, wherein the active agent is a mammalian target of rapamycin (mTOR) inhibitor or mitogen-activated protein kinase (MEK) inhibitor.
25. The composition according to any one of 1-11 wherein the active agent is a compound selected from the group consisting of a glucocorticoid, a heat shock protein inhibitor, a checkpoint inhibitor and a chemokine/chemokine ligand inhibitor.
26. The composition according to 25, wherein the glucocorticoid is fluocinolone acetonide.
27. The composition according to any one of 1-11, wherein the active agent is a compound for the treatment of glioma, a skin cancer or perineural invasion.
28. The composition according to any one of 1-27, wherein the active agent conjugate further comprises a linker that covalently bonds the active agent compound to the protein, peptide or peptidomimetic.
29. The composition according to 28, wherein the linker is selected from the group consisting of a bifunctional linker, a homobifunctional linker and a heterobifunctional linker.
30. The composition according to any one of 1-29, wherein the protein, peptide or peptidomimetic is bonded to the active agent through an internal amino acid residue.
31. The composition according to 30, wherein the amino acid residue is present at the surface of the protein, peptide or peptidomimetic.
32. The composition according to any one of 1-29, wherein the active agent is bonded to the N-terminal amino acid or C-terminal amino acid of the protein, peptide or peptidomimetic.
33. The composition according to any one of 28-32, wherein the linker is configured to bond to a mutated amino acid (e.g., substituted amino acid or non-natural amino acid) of the protein, peptide or peptidomimetic or to a reactive side chain of an amino acid in a native protein, peptide or peptidomimetic.
34. The composition according to any one of 28-33, wherein the linker is configured to bond to the protein, peptide or peptidomimetic through an ether linkage, an ester linkage, a carbamate linkage, an amide linkage, a disulfide linkage or an amino linkage.
35. The composition according to any one of 1-34, wherein the active agent compounds are covalently bonded to lysine residues of the protein, peptide or peptidomimetic.
36. The composition according to any one of 1-34, wherein the linker is covalently bonded to the active agent through a carbon on the active agent.
37. The composition according to any one of 1-34, wherein the linker is covalently bonded to the active agent through a non-carbon on the active agent.
38. The composition according to 37, wherein the linker is covalently bonded to the active agent through a nitrogen on the active agent.
39. The composition according to any one of 1-34, wherein the linker is covalently bonded to the active agent through a sulfur on the active agent.
40. The composition according to any one of 1-39, wherein the active agent conjugate is functionally capable of native protein dimerization to enable endocytosis and intraneuronal visualization of the dye.
41. The composition according to any one of 1-40, wherein the wherein the active agent conjugate facilitates binding of the protein, peptide or peptidomimetic that binds selectively to the neurotrophin receptor.
42. The composition according to any one of 1-40, wherein the wherein the active agent conjugate facilitates endocytosis.
43. The composition according to any one of 1-40, wherein the wherein the active agent conjugate facilitates intraneuronal transport.
44. A method of delivering an active agent conjugate to a nerve cell, the method comprising contacting a nerve cell with a composition according to any one of 1-43.
45. A method for delivering an active agent conjugate selectively into a nerve cell, the method comprising administering to a subject, a composition according to any one of 1-43.
46. The method according to 45, wherein the composition is administered intraocularly to the subject.
47. The method according to 45, wherein the composition is administered intracisternally to the subject.
48. The method according to 45, wherein the composition is administered intrathecally to the subject.
49. The method according to 45, wherein the composition is administered intravitreally to the subject.
50. The method according to 45, wherein the composition is administered topically.
51. The method according to 45, wherein the composition is by injection subcutaneously, intramuscularly, intravitreally, intracisternally, intrathecally, or a combination thereof.
52. A method comprising:

contacting an active agent compound with a protein, peptide or peptidomimetic that binds selectively to a neurotrophin receptor to generate a composition comprising an active agent conjugate, the active agent conjugate comprising one or more active agent compounds covalently bonded to the protein, peptide or peptidomimetic that binds selectively to a neurotrophin receptor, is endocytosed and enables intracellular transposition (e.g., retrograde axonal transport); and

isolating active agent conjugates from the composition having an average ratio of active agent compound to protein, peptide or peptidomimetic of 5 or less.

53. The method according to 52, wherein the method comprises isolating active agent conjugates from the composition having an average ratio of active agent compound to protein, peptide or peptidomimetic of 0.5 to 5.
54. The method according to any one of 52-53, wherein 95% or more of the active agent conjugates in the composition have an average ratio of active agent compound to protein, peptide or peptidomimetic of 5 or less.
55. The method according to any one of 52-53, wherein the average ratio of active agent compound to protein, peptide or peptidomimetic in the active agent conjugates in the composition is 3.2 or less.
56. The method according to any one of 52-54, wherein the method comprises isolating active agent conjugates from the composition having an average ratio of active agent compound to protein, peptide or peptidomimetic of 2 or less.
57. The method according to 56, wherein the method comprises isolating active agent conjugates from the composition having an average ratio of active agent compound to protein, peptide or peptidomimetic of from about 0.95 to about 1.85.
58. The method according to any one of 56-57, wherein 90% or more of the active agent conjugates in the isolated composition have an average ratio of active agent compound to protein, peptide or peptidomimetic of 2 or less.
59. The method according to any one of 56-57, wherein 95% or more of the active agent conjugates in the isolated composition have an average ratio of active agent compound to protein, peptide or peptidomimetic of 2 or less.
60. The method according to any one of 52-59, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.
61. The method according to 60, wherein the protein, peptide or peptidomimetic is a neurotrophic factor derived from a non-human animal.
62. The method according to 60, wherein the protein, peptide or peptidomimetic is a recombinant human neurotrophic factor.
63. The method according to 60, wherein the protein, peptide or peptidomimetic comprises a beta-subunit of NGF, rhNGF or rhBDNF, or a chimera constructed of NGF and BDNF, or a chimeric construct of rhNGF and any other neurotrophic factor, or a chimeric construct between any two other neurotrophic factors.
64. The method according to any one of 52-63, wherein the active agent is a dye.
65. The method according to 64, wherein the active agent is an organic dye or an inorganic dye.
66. The method according to 64, wherein the dye is selected from the group consisting of a bodipy dye, a coumarin dye, a rhodamine dye, an acridine dye, an anthraquinone dye, an arylmethane dye, a diarylmethane dye, a chlorophyll containing dye, a triarylmethane dye, an azo dye, a diazonium dye, a nitro dye, a nitroso dye, a phthalocyanine dye, a cyanine dye, an asymmetric cyanine dye, a quinon-imine dye, an azine dye, an eurhodin dye, a safranin dye, an indamin, an indophenol dye, a fluorine dye, an oxazine dye, an oxazone dye, a thiazine dye, a thiazole dye, a xanthene dye, a fluorene dye, a pyronin dye, a fluorine dye, a rhodamine dye, a phenanthridine dye, squaraines, bodipys, squarine roxitanes, naphthalenes, coumarins, oxadiazoles, anthracenes, pyrenes, acridines, arylmethines, or tetrapyrroles and a combination thereof.
67. The method according to 64, wherein the dye has an emission wavelength of 300 nm or more.
68. The method according to 64, wherein the dye is Alexa Fluor 488.
69. The method according to 64, wherein the dye is Dyomics DY-800.
70. The method according to 64, wherein the dye is ZW-800.
71. The method according to 64, wherein the dye is a compound having axial symmetry.
72. The method according to 64, wherein the dye is a compound having a plane of symmetry.
73. The method according to 64, wherein the dye is a compound that facilitates intraneuronal visualization of the dye, where intraneuronal visualization may be due to Trk receptor binding, endocytosis and retrograde axonal transport either in microtubules and/or in the nerve sheath encircling a nerve.
74. The method according to any one of 52-63, wherein the active agent is a small molecule active agent.
75. The method according to any one of 52-63, wherein the active agent is an anti-cancer agent.
76. The method according to any one of 52-63, wherein the active agent is a mammalian target of rapamycin (mTOR) inhibitor or mitogen-activated protein kinase (MEK) inhibitor.
77. The method according to any one of 52-63, wherein the active agent is a compound selected from the group consisting of a glucocorticoid, a heat shock protein inhibitor, a checkpoint inhibitor and a chemokine/chemokine ligand inhibitor.
78. The method according to 77, wherein the glucocorticoid is fluocinolone acetonide.
79. The method according to any one of 52-63, wherein the active agent is a compound for the treatment of glioma, a skin cancer or perineural invasion.
80. The method according to any one of 52-79, wherein the active agent conjugate further comprises a linker that covalently bonds the active agent compound to the protein, peptide or peptidomimetic.
81. The method according to 80, wherein the linker is selected from the group consisting of a bifunctional linker, a homobifunctional linker and a heterobifunctional linker.
82. The method according to any one of 52-81, wherein the protein, peptide or peptidomimetic is bonded to the active agent through an internal amino acid residue.
83. The method according to 82, wherein the amino acid residue is present at the surface of the protein, peptide or peptidomimetic.
84. The method according to any one of 52-83, wherein the active agent is bonded to the N-terminal amino acid or C-terminal amino acid of the protein, peptide or peptidomimetic.
85. The method according to any one of 81-84, wherein the linker is configured to bond to a mutated amino acid of the protein, peptide or peptidomimetic or to a reactive side chain of an amino acid in a native protein, peptide or peptidomimetic.
86. The method according to any one of 81-85, wherein the linker is configured to bond to the protein, peptide or peptidomimetic through an ether linkage, an ester linkage, a carbamate linkage, an amide linkage, a disulfide linkage or an amino linkage.
87. The method according to any one of 52-86, wherein the active agent compounds are covalently bonded to lysine residues of the protein, peptide or peptidomimetic.
88. The method according to any one of 52-86, wherein the linker is covalently bonded to the active agent through a carbon on the active agent.
89. The method according to any one of 52-86, wherein the linker is covalently bonded to the active agent through a non-carbon on the active agent.
90. The method according to 89, wherein the linker is covalently bonded to the active agent through a nitrogen on the active agent.
91. The method according to any one of 52-86, wherein the linker is covalently bonded to the active agent through a sulfur on the active agent.
92. The method according to any one of 52-91, wherein the active agent conjugate is functionally capable of native protein dimerization to enable endocytosis and intraneuronal visualization of the dye.
93. The method according to any one of 52-86, wherein the wherein the active agent conjugate facilitates binding of the protein, peptide or peptidomimetic that binds selectively to the neurotrophin receptor.
94. The method according to any one of 52-86, wherein the wherein the active agent conjugate facilitates endocytosis.
95. The method according to any one of 52-86, wherein the wherein the active agent conjugate facilitates intraneuronal transport.
96. A method for intraocularly delivering an active agent to a nerve cell, the method comprising contacting an eye of a subject with a composition comprising an active agent conjugate, the active agent conjugate comprising one or more active agent compounds covalently bonded to a protein, peptide or peptidomimetic that binds selectively to a neurotrophin receptor, is endocytosed and enables intracellular transposition (e.g., retrograde axonal transport).
97. The method according to 96, wherein the composition is topically contacted with a surface of the eye.
98. The method according to 97, wherein the composition is topically contacted with a corneal surface of the eye.
99. The method according to 96, wherein the composition is implanted into the eye of the subject.
100. The method according to 99, wherein the composition is implanted intravitreally into the eye of the subject.
101. The method according to any one of 96-100, wherein the composition is formulated for sustained release of the active agent conjugate.
102. The method according to 101, wherein the composition is formulated for release of the active agent conjugate into the eye over 7 days or longer.
103. The method according to 101, wherein the composition is formulated for release of the active agent conjugate into the eye over 1 month or longer.
104. The method according to any one of 96-100, wherein the composition is formulated for delayed release of the active agent conjugate.
105. The method according to any one of 96-100, wherein the composition is formulated for delayed immediate release of the active agent conjugate.
106. The method according to any one of 101-105, wherein the composition is formulated with a non-erodible polymer.
107. The method according to any one of 101-105, wherein the composition is formulated with a bio-erodible polymer.
108. The method according to any one of 96-107, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.
109. The method according to 108, wherein the protein, peptide or peptidomimetic is a neurotrophic factor derived from a non-human animal.
110. The method according to 109, wherein the protein, peptide or peptidomimetic is a recombinant human neurotrophic factor.
111. The composition according to 109, wherein the protein, peptide or peptidomimetic comprises a β-subunit of NGF, rhNGF or rhBDNF, or a chimera constructed of NGF and BDNF, or a chimeric construct of rhNGF and any other neurotrophic factor, or a chimeric construct between any two other neurotrophic factors.
112. The method according to any one of 96-111, wherein the protein, peptide or peptidomimetic is neurotrophic factor that binds tropomyosin kinase A (TrkA).
113. The method according to any one of 96-112, wherein the active agent is a dye.
114. The method according to 113, wherein the active agent is an organic dye or an inorganic dye.
115. The method according to 114, wherein the dye is selected from the group consisting of a bodipy dye, a coumarin dye, a rhodamine dye, an acridine dye, an anthraquinone dye, an arylmethane dye, a diarylmethane dye, a chlorophyll containing dye, a triarylmethane dye, an azo dye, a diazonium dye, a nitro dye, a nitroso dye, a phthalocyanine dye, a cyanine dye, an asymmetric cyanine dye, a quinon-imine dye, an azine dye, an eurhodin dye, a safranin dye, an indamin, an indophenol dye, a fluorine dye, an oxazine dye, an oxazone dye, a thiazine dye, a thiazole dye, a xanthene dye, a fluorene dye, a pyronin dye, a fluorine dye, a rhodamine dye, a phenanthridine dye, squaraines, bodipys, squarine roxitanes, naphthalenes, coumarins, oxadiazoles, anthracenes, pyrenes, acridines, arylmethines, or tetrapyrroles and a combination thereof.
116. The method according to 115, wherein the dye has an emission wavelength of 300 nm or more.
117. The method according to 115, wherein the dye is Dyomics DY-800.
118. The method according to 115, wherein the dye is Cy 7.5.
119. The method according to 115, wherein the dye is Cy 7.0.
120. The method according to 115, wherein the dye is Cy 5.5.
121. The method according to any one of 115-121, wherein the dye is a compound that facilitates intraocular visualization of dye binding to a nerve cell, where intraneuronal visualization may be due to Trk receptor binding, endocytosis and retrograde axonal transport either in microtubules and/or in the nerve sheath encircling a nerve.
122. The method according to any one of 96-112, wherein the active agent is a small molecule active agent.
123. The method according to 122, wherein the active agent is an anti-inflammatory agent.
124. The method according to 123, wherein the anti-inflammatory agent is a glucocorticoid.
125. The method according to 124, wherein the glucocorticoid is fluocinolone acetonide.
126. The method according to 122, wherein the active agent is antimicrobial compound.
127. The method according to 126, wherein the antimicrobial compound is an antiviral compound.
128. The method according to 122, wherein the active agent is an anti-cancer agent.
129. The method according to 122, wherein the active agent is a neuroprotective compound.
130. The method according to any one of claims 96-129, wherein the method further comprises determining the health of retinal ganglion cells in the eye of the subject.
131. The method according to any one of 96-130, wherein the active agent conjugate further comprises a linker that covalently bonds the active agent compound to the protein, peptide or peptidomimetic.
132. The method according to 131, wherein the linker is selected from the group consisting of a bifunctional linker, a homobifunctional linker and a heterobifunctional linker.
133. The method according to any one of 96-132, wherein the protein, peptide or peptidomimetic is bonded to the active agent through an internal amino acid residue.
134. The method according to 133, wherein the amino acid residue is present at the surface of the protein, peptide or peptidomimetic.
135. The method according to any one of 96-134, wherein the active agent is bonded to the N-terminal amino acid or C-terminal amino acid of the protein, peptide or peptidomimetic.
136. The method according to any one of 131-134, wherein the linker is configured to bond to a mutated amino acid of the protein, peptide or peptidomimetic or to a reactive side chain of an amino acid in a native protein, peptide or peptidomimetic.
137. The method according to any one of 131-134, wherein the linker is configured to bond to the protein, peptide or peptidomimetic through an ether linkage, an ester linkage, a carbamate linkage, an amide linkage, a disulfide linkage or an amino linkage.
138. The method according to any one of 96-137, wherein the active agent compounds are covalently bonded to lysine residues of the protein, peptide or peptidomimetic.
139. The method according to any one of claims 131-138, wherein the linker is covalently bonded to the active agent through a carbon on the active agent.
140. The method according to any one of claims 131-138, wherein the linker is covalently bonded to the active agent through a non-carbon on the active agent.
141. The method according to claim 140, wherein the linker is covalently bonded to the active agent through a nitrogen on the active agent.
142. The method according to any one of claims 131-138, wherein the linker is covalently bonded to the active agent through a sulfur on the active agent.
143. The method according to any one of claims 96-142, wherein the active agent compounds are covalently bonded to the protein, peptide or peptidomimetic through a disuccinimidyl carbonate linker.
144. A composition for use in a method according to any one of claims 96-143, the composition comprising an active agent conjugate, the active agent conjugate comprising one or more active agent compounds covalently bonded to a protein, peptide or peptidomimetic that binds selectively to a neurotrophin receptor.
145. A contact lens comprising a composition according to claim 144.
146. A composition comprising:
a first detectable label-biomolecule conjugate comprising a first detectable label covalently bonded to a protein, peptide or peptidomimetic that binds selectively to a neurotrophin receptor, is endocytosed and enables intracellular transposition (e.g., retrograde/anterograde axonal transport),
a second detectable label-biomolecule conjugate comprising a second detectable label covalently bonded to a biopolymer that binds selectively to neoplastic tissue.
   147. The composition according to 146, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.
   148. The composition according to 147, wherein the protein, peptide or peptidomimetic is a neurotrophic factor derived from a non-human animal.
   149. The composition according to 148, wherein the protein, peptide or peptidomimetic is a recombinant human neurotrophic factor.
   150. The composition according to 147, wherein the protein, peptide or peptidomimetic comprises a $-subunit of NGF, rhNGF or rhBDNF, or a chimeric construct of rhNGF and rhBDNF, or a chimeric construct of rhNGF and any other neurotrophic factor, or a chimeric construct between any two other neurotrophic factors.
   151. The composition according to any one of 146-150, wherein the protein, peptide or peptidomimetic is neurotrophic factor that binds tropomyosin kinase A (TrkA).
   152. The composition according to any one of 146-151, wherein each detectable label independently comprises a compound selected from the group consisting of a fluorophore, chromophore, enzyme, redox label, radiolabels, acoustic label, Raman (SERS) tag, mass tag, isotope tag, magnetic particle, microparticle and nanoparticle.
   153. The composition according to 152, wherein each detectable label is a dye.
   154. The composition according to 153, wherein each detectable label is an organic dye or an inorganic dye.
   155. The composition according to 154, wherein the dye is selected from the group consisting of a bodipy dye, a coumarin dye, a rhodamine dye, an acridine dye, an anthraquinone dye, an arylmethane dye, a diarylmethane dye, a chlorophyll containing dye, a triarylmethane dye, an azo dye, a diazonium dye, a nitro dye, a nitroso dye, a phthalocyanine dye, a cyanine dye, an asymmetric cyanine dye, a quinonimine dye, an azine dye, an eurhodin dye, a safranin dye, an indamin, an indophenol dye, a fluorine dye, an oxazine dye, an oxazone dye, a thiazine dye, a thiazole dye, a xanthene dye, a fluorene dye, a pyronin dye, a fluorine dye, a rhodamine dye, a phenanthridine dye, squaraines, bodipys, squarine roxitanes, naphthalenes, coumarins, oxadiazoles, anthracenes, pyrenes, acridines, arylmethines, or tetrapyrroles and a combination thereof.
   156. The composition according to 155, wherein the dye has an emission wavelength of 300 nm or more.
   157. The composition according to 155, wherein the dye is Alexa Fluor 680.
   158. The composition according to 155, wherein the dye is Dyomics DY-800.
   159. The composition according to 155, wherein the dye is Cy 7.5.
   160. The composition according to 155, wherein the dye is Cy 5.5.
   161. The composition according to any one of 146-160, wherein the first detectable label is a compound that facilitates peripheral nerve visualization.
   162. The composition according to any one of 146-160, wherein the first detectable label is a compound that facilitates intraneuronal visualization, where intraneuronal visualization may be due to Trk receptor binding, endocytosis and retrograde axonal transport either in microtubules and/or in the nerve sheath encircling a nerve.
   163. The composition according to any one of 146-162, wherein the biopolymer that binds selectively to a neoplastic tissue is a compound selected from the group consisting of a polypeptide, a nucleic acid and a polysaccharide.
   164. The composition according to 163, wherein the polypeptide is a peptidomimetic, a protein, an enzyme or an antibody.
   165. The composition according to 164, wherein the biopolymer that binds selectively to a neoplastic tissue is an antibody.
   166. The composition according to 165, wherein the biopolymer that binds selectively to a neoplastic tissue is a monoclonal antibody.
   167. The composition according to any one of 146-166, wherein the first detectable label-biomolecule conjugate further comprises a linker that covalently bonds the detectable label to the protein, peptide or peptidomimetic.
   168. The composition according to any one of 146-167, wherein the second detectable label-biomolecule conjugate further comprises a linker that covalently bonds the detectable label to the biopolymer that binds selectively to neoplastic tissue.
   169. The composition according to any one of 167-168, wherein the linker is selected from the group consisting of a bifunctional linker, a homobifunctional linker and a heterobifunctional linker.
   170. The composition according to any one of 146-169, wherein the protein, peptide or peptidomimetic is bonded to the active agent through an internal amino acid residue.
   171. The composition according to 170, wherein the amino acid residue is present at the surface of the protein, peptide or peptidomimetic.
   172. The composition according to any one of 167-171, wherein the linker is configured to bond to the N-terminal amino acid or C-terminal amino acid of the protein, peptide or peptidomimetic.
   173. The composition according to any one of 167-172, wherein the linker is configured to bond to a mutated amino acid of the protein, peptide or peptidomimetic.
   174. The composition according to any one of 167-172, wherein the linker is configured to bond to the protein, peptide or peptidomimetic through an ether linkage, an ester linkage, a carbamate linkage, an amide linkage, a disulfide linkage or an amino linkage.
   175. The composition according to any one of 146-174, wherein the first detectable label is covalently bonded to lysine residues of the protein, peptide or peptidomimetic.
   176. The composition according to any one of 146-175, wherein the first detectable label is covalently bonded to the protein, peptide or peptidomimetic through a carbon on the protein, peptide or peptidomimetic.
   177. The composition according to any one of 146-176, wherein the first detectable label is covalently bonded to the protein, peptide or peptidomimetic through a non-carbon on the protein, peptide or peptidomimetic.
   178. The composition according to 177, wherein the first detectable label is covalently bonded to the protein, peptide or peptidomimetic through a nitrogen on the protein, peptide or peptidomimetic.
   179. The composition according to 177, wherein the first detectable label is covalently bonded to the protein, peptide or peptidomimetic through a sulfur on the protein, peptide or peptidomimetic.
   180. The composition according to any one of 168-179, wherein the second detectable label-biomolecule conjugate comprises an antibody that binds selectively to neoplastic tissue and the linker is configured to bond to the N-terminal amino acid or C-terminal amino acid of the antibody.
   181. A method comprising contacting a tissue of a subject with a composition according to any one of 146-180.
   182. The method according to 181, wherein the composition is administered to the subject.
   183. The method according to 182, wherein the composition is administered topically, topically intra-operatively, intravenously, intraocularly, intracisternally, intrathecally, intravitreally, or by injection subcutaneously, intramuscularly, intravitreally, intracisternally, intrathecally, or a combination thereof.
   184. The method according to any one of 181-183, wherein the composition is contacted with the tissue of the subject during surgery.
   185. The method according to 184, wherein the surgery comprises resection of at least a part of the neoplastic tissue.
   186. A composition comprising:

a nerve imaging agent comprising a first fluorescent compound covalently bonded to a protein, peptide or peptidomimetic that binds selectively to a neurotrophin receptor; and

a tumor imaging agent comprising a second fluorescent compound covalently bonded to an antibody that binds selectively to malignant neoplastic tissue.

187. The composition according to claim 186, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.
188. The composition according to any one of 186-187, wherein the first fluorescent compound, second fluorescent compound or combination thereof is selected from the group consisting of Alexa Fluor 680, Dyomics DY-800, Cy 7.5 and Cy 5.5.
189. The composition according to any one of 186-188, wherein the antibody is Panitumumab.
190. A method comprising contacting a tissue of a subject undergoing surgery with a composition comprising:

a nerve imaging agent comprising a first fluorescent compound covalently bonded to a protein, peptide or peptidomimetic that binds selectively to a neurotrophin receptor; and

a tumor imaging agent comprising a second fluorescent compound covalently bonded to an antibody or antibody fragment that binds selectively to malignant neoplastic tissue,

wherein the nerve imaging agent and tumor imaging age are configured to provide a visual aid during surgery.

191. The method according to 190, wherein the surgery is for removing malignant neoplastic tissue from the subject.
192. The composition according to any one of 190-191, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.
193. The method according to any one of 190-192, wherein the first fluorescent compound, second fluorescent compound or combination thereof is selected from the group consisting of Alexa Fluor 680, Dyomics DY-800, Cy 7.5 and Cy 5.5. 194. The method according to any one of 190-193, wherein the antibody is Panitumumab.
195. The method according to any one of 190-193, wherein the antibody is folate receptor alpha (folate receptor-α).

### Examples

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Neurotrophins as Neuronal Targeting Vectors

Neurotrophins (NTs) described herein include soluble peptides, Nerve Growth Factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7, glial cell-derived neurotrophic factor (GDNF), and ciliary neurotrophic factor (CNTF). In the developing mammal, NTs may direct development and differentiation of specific tissues and organs of the nervous system, i.e., retinal development is controlled by NGF, BDNF, and NT-3. NTs promote cell growth, survival and differentiation in several classes of neurons. NTs have been identified as being associated with neuronal survival. Each of the family of NTs is selective for a certain high-affinity receptor. Trk receptors are encoded by Trk genes TrkA, TrkB, and TrkC. All NTs also bind to the low affinity p75 neurotrophin receptor, p75-NTR. NTs were selected in this embodiment as neuronal delivery vectors based in part on the known selectivity and transport characteristics. NTs are endocytosed selectively by neurons at the site of peripheral injection or at the site of topical application by binding to high-affinity tropomyosin kinase receptors (TrkA for NGF, TrkB for BDNF, TrkC for NT-3) which are expressed on the distal ends of specific neuronal populations (e.g., dendites) as well as expressed more widely on cells of the immune system. The prevailing hypothesis is that the NT/Trk complex is moved intraneuronally via signaling endosomes in microtubules via retrograde axonal transport to the neuronal cell body, for example, to the Dorsal Root Ganglia (DRG) in the peripheral nervous system (PNS). It is also understood that the NT/Trk complex may be moved in the opposite direction via anterograde axonal transport. When administered exogenously, NTs are well-tolerated at low doses and therefore considered safe. NTs have been found to be not immunogenic, which is a feature that distinguishes NTs from viral vectors. NTs are localized to the neurons (e.g., dendrites) associated with the site of exogenous administration and distal proximity. NGF is degraded by lysosomal proteases. The compounds of compositions disclosed herein should not jump trans-synaptically from the PNS to the brain.

The subject compositions provided herein demonstrate that conjugates of interest may localize to specific neuronal populations to treat diseases, an effect which may improve the Therapeutic Index of otherwise systemically toxic agents due to their localization and associated reduced requirement for dose, e.g., fluocinolone acetonide (FA), a glucocorticoid with systemic dose-limiting toxicities; selectively treat pathological defects i.e. Trk defects that affect those neurons; and/or treat the microtubules and/or other molecular pathophysiological processes i.e. signaling endosomes involved in the retrograde or anterograde axonal transport of specific neuronal populations. The following experiments discuss:
Structural considerations for neurotrophins, and their selectivity due to their high affinity for specific receptors expressed on the distal ends of certain neuronal populations;
The clinical safety of rhNGF and rhBDNF;
Mechanisms of intraneuronal delivery via retrograde axonal transport, wherein mechanisms for anterograde axonal transport are known but are not discussed here;
Fluorescent dyes that may be considered for conjugation; and
Evidence of limitations of the conjugation of NT-small molecule compounds (SMCs, where NT-SMC expresses the ratio of SMC to protein) to effect binding, endocytosis, and retrograde axonal transport.

### Neurotrophins: Structural Considerations

The crystal structure has been fully elucidated for NGF. The precursor form of NGF (pro-NGF) is activated into biologically functional NGF following its cleavage to the active mature form of the molecule, resulting in a single chain of approximately 13 kDa weight. This monomeric form naturally and non-covalently combines to form a dimeric structure of 26,518 Da. BDNF follows a similar process, resulting in a non-covalent, dimeric, biologically functional mature form of approximately 26 kDa.

The structure of NGF includes amino acid residues that are physically distant from regions involved in binding to TrkA receptors. Cellular events required to bind and transport NGF retrogradely require the high affinity receptor TrkA. BDNF is roughly 55% homologous with NGF, with five of 11 lysine residues conserved between them; only two of the 11 lysines of BDNF are physically near TrkB sites. It is a dimer of two identical 119 amino acid subunits of MW 28 kDa held together by strong hydrophobic interactions. When BDNF binds to TrkB at distal ends, BDNF/TrkB complexes are internalized into signaling endosomes that are moved retrogradely by dynein motors along axonal microtubules. Similar to NGF-TrkA, the BDNF-TrkB complex is transported retrogradely to sensory and central (motor) neuronal cell bodies. There is other evidence that the NGF-TrkA and BDNF-TrkB complexes may be moved via anterograde axonal transport. Structural considerations as well as mutagenesis data suggest that BDNF interacts with TrkB very similarly to how NGF interacts with TrkA.

NTs localize to the neuronal populations associated with its proximate Trk receptor group in a phenomenon known as differential distribution. NTs are selective due to their distribution of Trk receptors.

Clinical Safety of Neurotrophins rhNGF and rhBDNF

Unmodified NTs are non-immunogenic. rhNGF can be formulated for ocular topical administration (e.g., eye drops given 20 μg/ml 6×QD for 8 weeks) to treat neurotrophic keratitis. rhNGF was well-tolerated when administered sub-cutaneously (SQ) up to a Maximum Tolerated Dose (MTD) of 1 μg/kg. rhNGF has been evaluated as a therapeutic agent in the treatment of intracerebroventricular (ICV) administration for Alzheimer's disease, multiple sclerosis, pain associated with multiple sclerosis, and other forms of global pain. rhBDNF has been evaluated when administered subcutaneously and intrathecally for the treatment of amyotrophic lateral sclerosis (ALS), depression, Post-Traumatic Stress Disorder (PTSD), and glaucoma.

### Compositions of Nerve Growth Factor (NGF)-Fluocinolone Acetonide Conjugates and Compositions of of Brain-Derived Neurotrophic Factor (BDNF)-Fluocinolone Acetonide Conjugates

As an example of an intracellularly active small molecule compound (SMC), the glucocorticoid fluocinolone acetonide (FA) was conjugated using a heterobifunctional linker to rhNGF and rhBDNF, both of which constructs exerted analgesic (anti-inflammatory) activity in vivo. Dorsal Root Ganglia (DRG) in diseases of the peripheral nervous system (PNS) play a fundamental role in chronic pain generally. NGF and BDNF localize due to their selectivity for distinct populations of peripheral neurons by binding to TrkA and TrkB expressed largely in nociceptors and proprioceptors, respectively. In treating forms of pain in which the DRG are involved, either NGF and/or BDNF should deliver intracellularly active FA by axonal transport to subpopulations of sensory neuron cell bodies in DRG and via their central synaptic terminals, into the dorsal horn of the spinal cord. Since TrkB is highly expressed in the neurofilament-1-expressing subpopulation of low-threshold mechanosensory neurons in the DRG, peripherally injected BDNF should deliver FA to the DRG. In treating pain, FA may alter neuronal excitability to be released or diffuse, acting on nearby inflammatory cells e.g. microglia, astrocytes, or satellite glia. rhNGF is stable in plasma for up to 4 days at 37° C.

In vitro systems that use primary neurons express Trk receptors on distal ends were used so that both survival and Trk binding activity may be assessed following synthetic manipulation in producing conjugates.

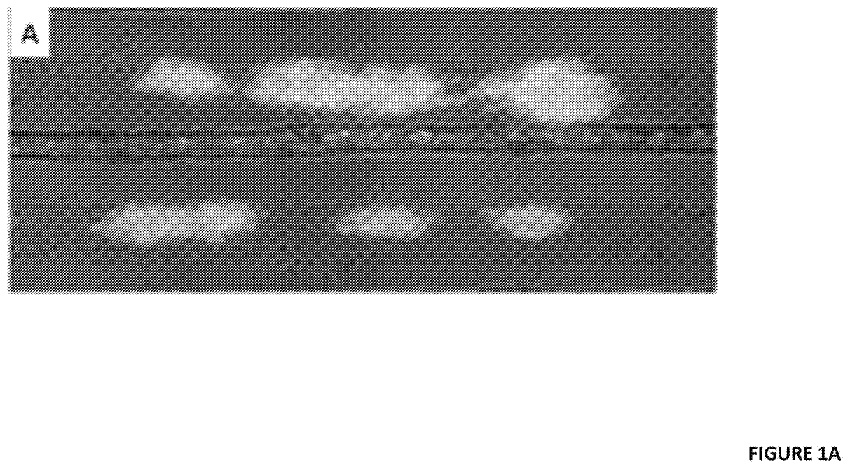

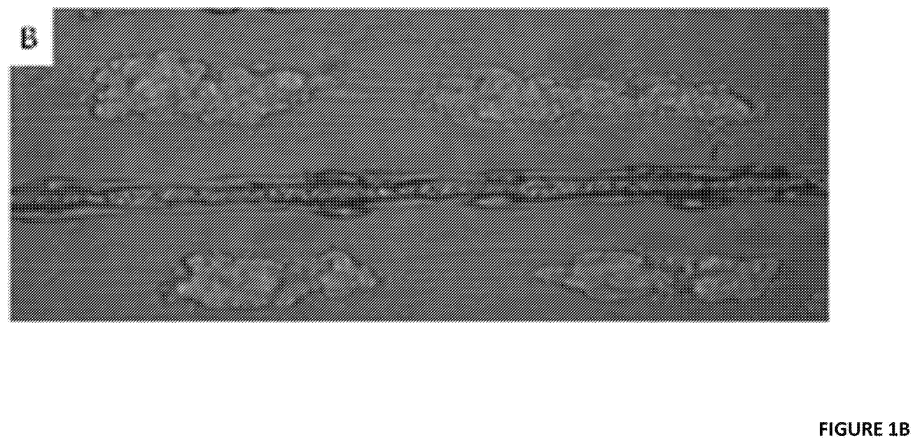

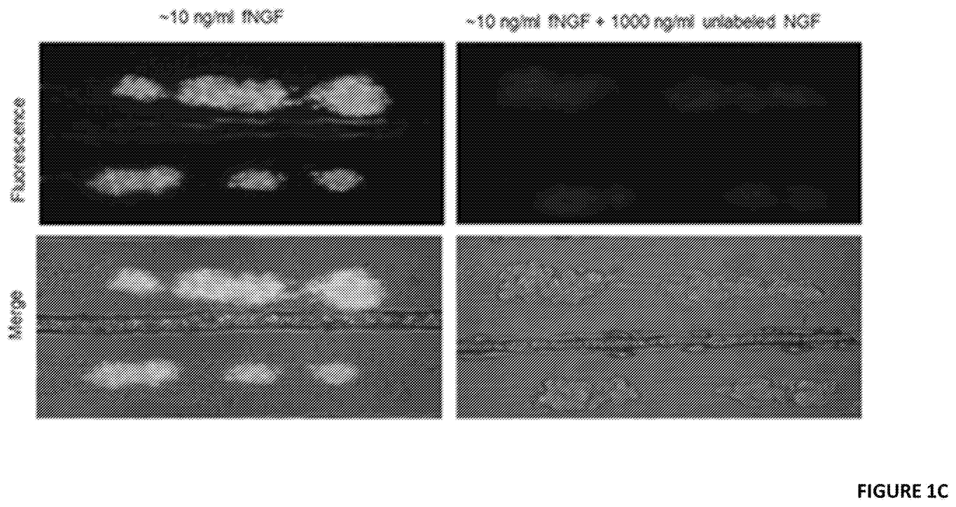

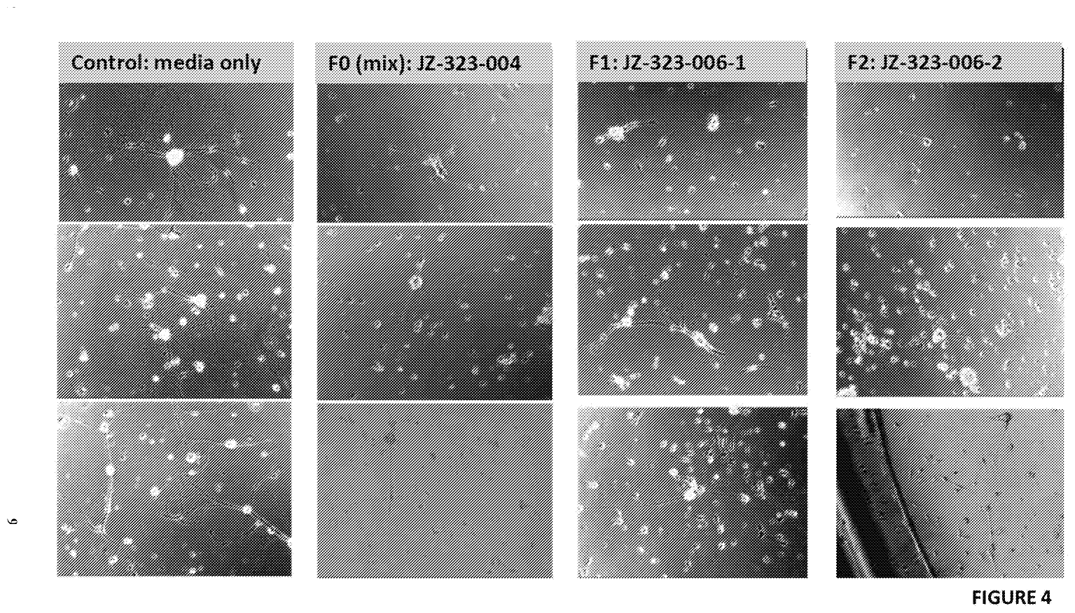

Confirm Trk receptor binding. The fluorescent dye Alexa Fluor 488 attached to murine NGF (488-mNGF, or fNGF) was endocytosed at the distal ends of peripheral (superior cervical ganglia, SCGs) neurons via a TrkA-mediated mechanism. Receptor binding activity where up to 100× unmodified mNGF is co-administered with the Trk binding agent was determined. 488-mNGF (FIG. 1A) is shown in cell bodies, after binding to TrkA, endocytosis and transport to the central chamber of the in vitro system. In FIG. 1B, TrkA was confirmed here by adding 1000 ng/ml unmodified mNGF to compete with 100 ng/ml 488-mNGF for the same TrkA receptors. FIG. 1C: Neurons in compartmented cultures were given approximately 10 ng/mL fNGF (as determined by neuronal survival bioassay), or 10 ng/mL fNGF+100× excess unlabelled NFG (1000 ng/mL) in the distal axon compartments. 24 hours later fluorescence images of the cell bodies were obtained (FIG. 1C). Accumulation of fluorescence in the cell bodies was effectively inhibited when 100× excess unlabelled NGF was added in addition to the fNGF. Uptake of fNGF is a specific, TrkA receptor-mediated process. Alexa Fluor requirement for DAR<4 per mouse NGF monomer. Fluorescent dye Alexa Fluor 488, which due to its size and hydrophobicity is attached to NGF without using a spacer of any length binds to TrkA, is endocytosed after binding, and is transported retrogradely to the neuronal cell body. In FIG. 4, we show in vitro results for a “mixed DAR” 800-rhNGF of 1.64 which is expressed as an average of 1-4 dye molecules per monomer using standard analytical methods mass spectroscopy (MS) or high performance liquid chromatography (HPLC), in each case where the NT involved is a dimer. Both FIG. 4 and FIG. 1C also demonstrate that 488-murine NGF sustained the survival of neurons, in this assay using superior cervical ganglia (SCGs).
Survival. Superior cervical ganglia (SCG) neurons in primary cultures were used in an established assay first in compartmented cultures and adapted later to grow and test neurons in microfluidic devices. SGC neurons were harvested from E-21 Sprague Dawley rat pups. In all studies conducted in microfluidic devices, the labeled NGF, either 800-rhNGF or FA-rhNGF, showed similar bioavailability as the unlabeled NGF, i.e., the TrkA binding sites remained intact after synthetic manipulation.

Labeled BDNF test articles, either 800-rhBDNF or FA-rhBDNF, were also assessed to as to whether the compositions would sustain neuronal survival by plating conjugates in Retinal Ganglion Cells (RGCs). RGCs were isolated from P6 rats by sequential immune-panning. Retinas are isolated and enzymatically digested by papain followed by mechanical dissociation into a single-cell suspension. Cells are negatively panned to remove macrophages and other nonspecifically adhering cells and then positively panned for RGCs using the Thy1.1 T11D7 hybridoma supernatant. Labeled rhBDNF has similar activity as unlabeled BDNF, i.e., TrkB binding is intact.

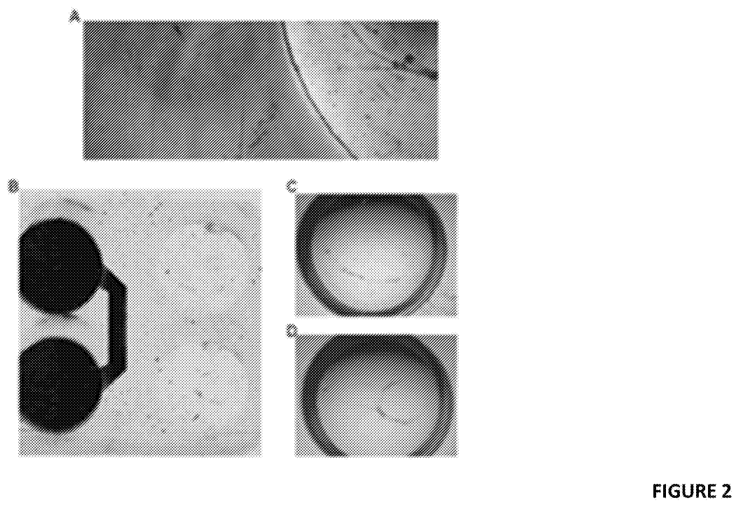

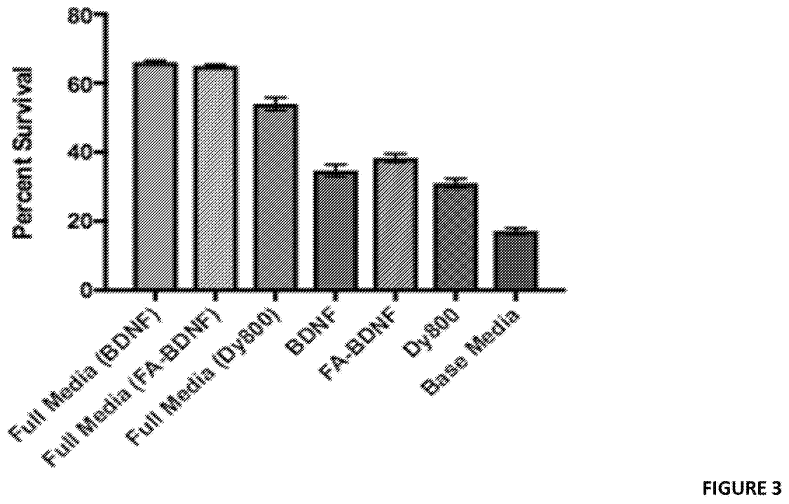

Increasing conjugation leads to decreased protein conjugate solubility. A construct where the active agent has a greater molecular weight and less hydrophilic Dyomics DY-800 Near InfraRed (NIR) dye was attached directly to recombinant human NGF (rhNGF) (FIG. 2). In subsequent studies, we purified DAR sub-species using a (Waters column) reverse phase HPLC (RP-HPLC). Each DAR sub-species showed varying levels of protein conjugate solubility with increasing levels of conjugation causing large increases in precipitation. FIG. 2A depicts superior cervical ganglia (SCG) neurons collected from E-21 Sprague Dawley rat pups and plated on one side of Xona microfluidic chambers. Cultures were maintained until the axons extended through the channels in the microfluidic chambers to the side without cells plated. FIG. 2B depicts 800-rhNGF, 30 ng/mL was added to the wells where the terminal (distal) axons had extended. 48 hours after addition of 800-rhNGF to distal ends in left chambers, chambers were imaged on a LI-COR Odyssey scanner. 800 Near InfraRed (NIR) labelled cell bodies can be visualized, indicating that the modified rhNGF compound has been retrogradedly transported. FIGS. 2C and 2D depict brightfield images of wells containing cell bodies, showing improved visualization of cell clumps for comparison to images containing 800 NIR cell bodies.
Neuronal Survival Confirms TrkB Binding Activity is Intact after Synthetic Manipulation.
FIG. 3 depicts that after synthetic manipulation, the TrkB binding activity of two constructs, NIR 800-rhBDNF where the NIR was directly attached to rhBDNF and FA-rhNGF where the glucocorticoid fluocinolone acetonide (FA) was conjugated to rhBDNF using a heterobifunctional linker, both sustained neuronal survival in primary neurons of Retinal Ganglion Cells (RGCs).

### Ratio of Active Agent to Protein in Conjugate Compositions

Analytics of 800-rhNGF DAR

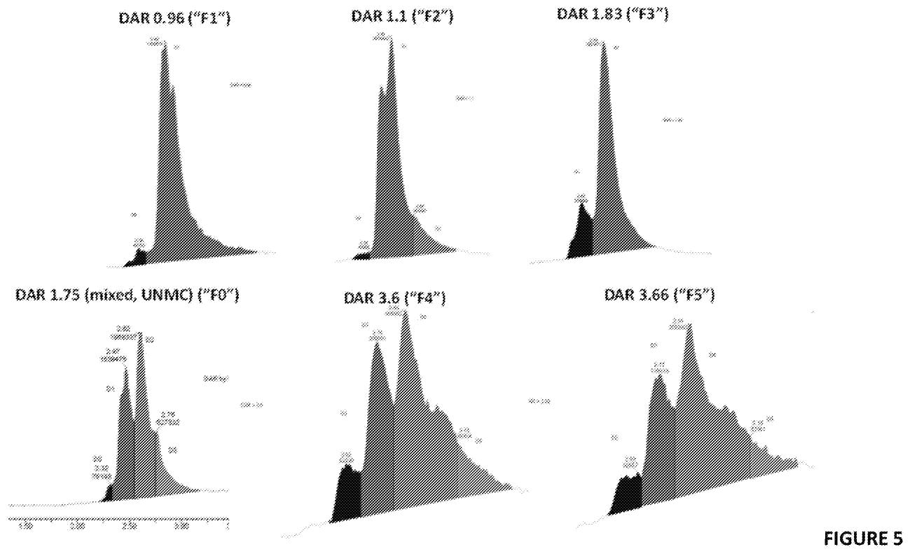

Despite the number of lysines which are available for protein labeling and/or crosslinker attachment, where lysines are physically distinct from Trk receptors, in this embodiment only NT-dyes where the ratio of active agent to NT (DAR) having an average ratio of ≤3.2 per monomer could sustain neuronal survival. DAR analytics were calculated using Reverse Phase High Performance Liquid Chromatography (RP-HPLC) and/or mass spectroscopy (MS). Using MS DAR analytics throughout for a fluorescent dye in the 800 region attached to rhNGF (800-rhNGF), as shown in FIG. 4 reading left to right, the leftmost panel is control, then F0, mixed or average DAR=1.64; F1=0.96 DAR; F2=1.1 DAR; and F3=1.83 DAR). All these DAR species sustained neuronal survival. In addition, the F0 “mixed DAR” of 1.64 was successful in vivo in localizing to and imaging target nerves. Higher DARs of F4=3.6 and F5, =3.66 failed to sustain neuronal survival. It was not possible to synthesize higher DARs, these “crashed out” before conjugation as shown by Reverse Phase High Performance Liquid Chromatography analysis. (FIG. 5)

Analytics of 800-rhBDNF

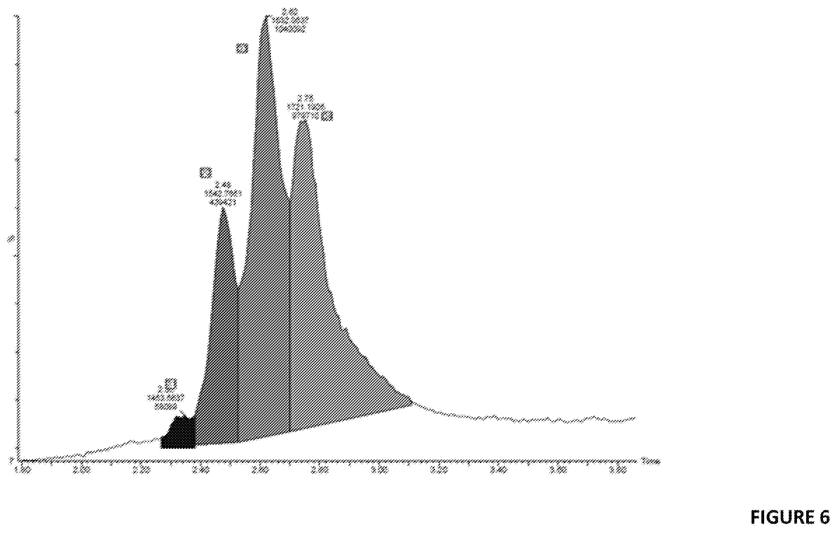

High Performance Liquid Chromatography and mass spectrometry was used to quantify the number of dye molecules attached or crosslinked to rhBDNF (i.e., the ratio of fluorescent dye in the 800 region to rhBDNF). In this embodiment, the average ratio of about 3.1 per monomer (see FIG. 6) which exhibit Trk binding, intraneuronal uptake and sustained neuronal survival (see FIG. 3).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A composition comprising an active agent conjugate, the active agent conjugate comprising one or more active agent compounds covalently bonded to a protein, peptide or peptidomimetic that binds selectively to a neurotrophin receptor,
wherein the average ratio of active agent compound to protein, peptide or peptidomimetic in the active agent conjugates in the composition is 5 or less.

2. The composition according to claim 1, wherein the average ratio of active agent compound to protein, peptide or peptidomimetic in the active agent conjugate in the composition is 3 or less.

3. The composition according to any one of claims 1-2, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.

4. The composition according to any one of claims 1-3, wherein the active agent is a dye.

5. The composition according to claim 4, wherein the dye is selected from the group consisting of Alexa Fluor 488, Alexa Fluor 670, Alexa Fluor 680, Dyomics DY-800, Cy 7.5, Cy 5.5, Dyomics DY-800, and ZW-800.

6. The composition according to any one of claims 1-3, wherein the active agent is a small molecule active agent.

7. The composition according to claim 6, wherein the active agent is an anti-cancer agent.

8. The composition according to claim 6, wherein the active agent is fluocinolone acetonide.

9. The composition according to any one of claims 1-8, wherein the active agent conjugate further comprises a linker that covalently bonds the active agent compound to the protein, peptide or peptidomimetic, wherein the linker is a bifunctional linker.

10. The composition according to any one of claims 1-9, wherein the active agent conjugate facilitates one or more of:
a) endocytosis;
b) axonal transport; and
c) pharmacological activity in the neuronal cell body.

11. A method comprising:
contacting an active agent compound with a protein, peptide or peptidomimetic that binds selectively to a neurotrophin receptor to generate a composition comprising an active agent conjugate, the active agent conjugate comprising one or more active agent compounds covalently bonded to the protein, peptide or peptidomimetic that binds selectively to a neurotrophin receptor; and
isolating active agent conjugates from the composition having an average ratio of active agent compound to protein, peptide or peptidomimetic of 5 or less.

12. The method according to claim 11, wherein the method comprises isolating active agent conjugates from the composition having an average ratio of active agent compound to protein, peptide or peptidomimetic of 3 or less.

13. A method for intraocularly delivering an active agent to a nerve cell, the method comprising contacting an eye of a subject with a composition comprising an active agent conjugate, the active agent conjugate comprising one or more active agent compounds covalently bonded to a protein, peptide or peptidomimetic that binds selectively to a neurotrophin receptor.

14. A composition comprising:
a first detectable label-biomolecule conjugate comprising a first detectable label covalently bonded to a protein, peptide or peptidomimetic that binds selectively to a neurotrophin receptor,
a second detectable label-biomolecule conjugate comprising a second detectable label covalently bonded to a biopolymer that binds selectively to neoplastic tissue.

15. A composition according to claim 14, comprising:
a nerve imaging agent comprising a first fluorescent compound covalently bonded to a protein, peptide or peptidomimetic that binds selectively to a neurotrophin receptor; and
a tumor imaging agent comprising a second fluorescent compound covalently bonded to an antibody that binds selectively to a receptor of a malignant neoplastic tissue.
